(19) United States
(12) Patent Application Publication
(10) Pub. No.: US 2010/0316628 A1
(43) Pub. Date: Dec. 16, 2010

(54) AGENTS AND METHODS FOR TREATING RESPIRATORY DISORDERS

(75) Inventors: Sylvie Breton, Arlington, MA (US); Dennis Brown, Natick, MA (US); Wai Chi Shum, Allston, MA (US); Nicolas Da Silva, Winchester, MA (US)

Correspondence Address: DAVID S. RESNICK, NIXON PEABODY LLP, 100 SUMMER STREET, BOSTON, MA, 02110-2131, US

(73) Assignee: THE GENERAL HOSPITAL CORPORATION, Boston, MA (US)

(21) Appl. No.: 12/634,432

(22) Filed: Dec. 9, 2009

Related U.S. Application Data
(60) Provisional application No. 61/120,892, filed on Dec. 9, 2008.

(51) Int. Cl. A61K 39/395; A61P 11/00; A61P 37/08; A61P 11/06; A61K 38/02; A61K 38/16; A61K 31/7088; A61K 31/713; A61K 31/4166; A61K 31/416; A61K 31/555; A61K 31/28

(52) U.S. Cl. 424/130.1; 514/1.7; 514/44 R; 514/44 A; 514/401; 514/403; 424/178.1; 514/185; 514/492

(57) ABSTRACT

The present invention relates to a novel method of preventing and/or treating respiratory disorders and respiratory-related complications in a subject by inhibiting the release of nitric oxide from basal cells in the respiratory tract.

### RELATED APPLICATIONS

This application claims benefit and priority under 35 U.S.C. §119(e) of the U.S. Provisional Application No. 61/120,892, filed Dec. 9, 2008, the content of which is herein incorporated by reference in its entirety.

### GOVERNMENT SUPPORT

This invention was made with government support under NIH grants no.: HD40793, DK38452, DK42956, and NCRR P41 RR001395. The government has certain rights in this invention.

### FIELD OF THE INVENTION

The invention relates to methods and compositions for treating respiratory disorders.

### BACKGROUND OF THE INVENTION

Epithelial cells have developed complex mechanisms that allow them to detect both apical and basolateral stimuli, and modulate their function in response to physiological demands. The different cell types that comprise specific epithelia must work in a concerted manner to coordinate their barrier function. Previous studies have largely focused on the morphologically dominant epithelial cells in several tissues, whereas basal cells that are nestled beneath these epithelial cells have remained mostly enigmatic. These cells are believed to be restricted to the basal region of pseudostratified epithelia where they may function as stem cells (Ford and Terzaghi-Howe, 1992; Hajj et al., 2007; Ihrler et al., 2002; Lavker et al., 2004; Leung et al., 2007; Rizzo et al., 2005), and participate in basolateral signaling (Evans et al., 2001; Hermo and Robaire, 2002; Ihrler et al., 2002; Leung et al., 2004; van Leenders and Schalken, 2003).

The epididymal epithelium, which connects the testis to the vas deferens, forms a tight blood/epididymis barrier and establishes an optimal luminal environment for the maturation and storage of spermatozoa (Hermo and Robaire, 2002; Hinton and Palladino, 1995). Male fertility is partially regulated via the renin-angiotensin system (RAS) located in the tubule lumen (Hagaman et al., 1998; Krege et al., 1995; Leung and Sernia, 2003). Both angiotensin II (ANGII) type 1 and type 2 receptors (AGTR1 and AGTR2) are expressed in the epididymal epithelium (Leung et al., 1997; Leung and Sernia, 2003; Saez et al., 2004). In the kidney collecting duct, which bears a striking functional and cellular resemblance to the epididymal tubule, ANGII increases proton secretion in specialized intercalated cells (Pech et al., 2008; Rothenberger et al., 2007). Similar cells, called clear cells, are also present in the epididymis where they are responsible for luminal acidification (Breton et al., 1996; Brown et al., 1992), which is essential for keeping sperm dormant during maturation and storage (Hinton and Palladino, 1995; Pastor-Soler et al., 2005).

Nitric oxide is produced by the lung and is normally present in the exhaled air of healthy people. However, its concentration increases significantly in inflammatory disorders of the lung such as asthma and COPD. The exact cellular source of NO in health and disease is not completely elucidated, although epithelial cells have been shown to be contributors in addition to nerves and blood vessels. NO is an important mediator of inflammation, in addition to being a vasodilator. The inflammatory property of NO has led to the proposal that inhibitors of nitric oxide synthases might help treat asthma. While NO production did significantly decrease in patients treated with various NOS inhibitors, including “specific” inhibitors of the inducible NOS, no significant positive physiological benefits were observed in these patients.

Because NO is involved in the regulation of a variety of physiological processes, it is crucial to target the cell type in which NO production would initiate the disease, while preserving the functional integrity of the surrounding tissues.

### SUMMARY OF THE INVENTION

In one aspect the invention is directed to a method for treating respiratory disorders. The method of treatment involves inhibiting release of nitric oxide (NO) from a basal cell. The respiratory disorder is allergic or non-allergic. In some embodiments, the respiratory disorder is selected from the group consisting of atopic asthma, non-atopic asthma, emphysema, bronchitis, chronic obstructive pulmonary disease, sinusitis and allergic rhinitis. One embodiment of the invention is a method of treating respiratory disorder comprising the step of administering to a subject a nitric oxide inhibitor.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

The patent or application file contains at least one drawing executed in color. Copies of this patent or patent application publication with color drawing(s) will be provided by the Office upon request and payment of necessary fee.

FIGS. 1A-1J show basal cells send projections towards the lumen. FIG. 1A shows rat corpus epididymidis stained for COX1 (green). Higher magnification is shown in inset. Arrows indicate basal cells that extend towards the lumen. Bars: 50 μm, 5 μm (inset). FIG. 1B shows 3D-reconstruction of cauda epididymidis labeled for COX1 (green) showing two basal cells reaching towards the lumen (arrows, Bar: 8 μm). FIG. 1C shows the oblique section of cauda epididymidis stained for Cldn1 (green). Basal cell body projections infiltrating between epithelial cells are seen as small dots (arrows). The inset shows two basal cells with intracellular COX1 (red) and membrane-bound Cldn1 (green, Bars: 20 μm, 5 μm (inset). FIG. 1D shows 3D-reconstruction of corpus epididymidis double-stained for COX1 (red) and Cldn1 (green) showing several basal cell extensions reaching out to the lumen (Bar: 8 μm). FIG. 1E shows quantification of the total number of basal cells in different regions of the epididymis and the proximal vas deferens. Data are represented as mean±SEM. No significant differences were detected between the regions. p-IS: proximal initial segment, d-IS: distal initial segment, Inter-zone: intermediate zone, pCPT: proximal caput, dCPT: distal caput, pCPS: proximal corpus, mCPS: middle corpus, dCPS: distal corpus, pCD: proximal cauda, mCD: middle cauda, dCD: distal cauda, VD: proximal vas deferens. FIG. 1F shows percentage of basal cells detected with their body projection reaching the apical pole of the epithelium (open bars) and percentage of basal cells detected at the apical border (solid bars). Data are represented as mean±SEM. Number of cells reaching the apical pole/total number of basal cells are indicated above the bars. FIG. 1G shows rat trachea stained for COX1 (red) and tubulin (green). Arrow shows a COX1-positive basal cell that extends towards the lumen (arrow). The cilia of adjacent ciliated cells are labeled for tubulin. Some unidentified COX1-positive cells were also detected (Bar: 15 μm. FIG. 1H shows 3D-reconstruction of a trachea section double-stained for COX1 (red) and ZO1 (green) showing a basal cell reaching the apical border of the epithelium (arrow). Unidentified COX1-positive cells were also detected (Bar: 5 μm). FIG. 1I shows rat coagulating gland stained for COX1 (green). Several basal cells extend towards the lumen (arrows). The inset shows a COX1-positive basal cell (green) visualized by DIC (arrows, Bars: 15 μm, 5 μm (inset)). FIG. 1J shows human epididymis 5 μm section stained for COX1 (green). Numerous basal cells are seen in the basal region of the epithelium. Some basal cells are also detected, even on this thinner section, with their body projections reaching the apical region of the epithelium (arrows). Lu: lumen, IT: interstitium. In some of FIGS. 1A-1J, nuclei and spermatozoa were stained in blue with DAPI.

FIGS. 2A-2H show basal cells cross TJs. FIG. 2A (panels labeled A′, A″ and A′″) shows three different rotations of a 3D reconstruction of an epididymis section stained for Cldn1 (red) and ZO1 (green). Basal cells reach the TJs at the intersection between three epithelial cells (arrows, Bar: 10 μm). FIG. 2B shows conventional microscopy image of one basal cell (stained for Cldn1 in red) forming a tight junction (stained for ZO1 in green) with adjacent principal cells (arrow). A clear cell expressing apical V-ATPase (blue) is seen (arrowhead). The nuclei are also detected in blue (DAPI) (Bar: 5 μm). FIGS. 2C-2F show body projections of basal cells showing different patterns of interaction with TJs. FIG. 2C shows no co-localization between Cldn1 and ZO1 (arrow). FIG. 2D shows partial co-localization of Cldn1 with ZO1 (arrows). FIG. 2E shows basal cell that penetrates the TJ (arrow). FIG. 2F shows basal cell showing ZO1-stained TJ (green) with adjacent principal cells (arrows). FIG. 2G (panels labeled G′, G″ and G′″) shows rotations of a 3D reconstruction of epididymis stained for Cldn1 (green) and F-actin (red). A Cldn1-positive basal cell reaches the luminal side (arrow) between F-actin-labelled principal cells (Bar: 5 μm). FIG. 2H (panels labeled H′ and H″) shows enface view visualized by DIC and immunofluorescence Cldn1 labeling (green). The dotted lines in panel H″ indicate the junctions between epithelial cells. Arrows show the tricellular corners between epithelial cells. One corner is occupied by a Cldn1-positive basal cell.

FIGS. 3A-3E show expression of AGTR2 in basal cells. FIG. 3A (panels labeled A′, A″ and A′″) shows three examples of AGTR2 (green) and V-ATPase (red) labeling in cauda epididymidis. Arrows indicate AGTR2-labelled basal cells that send projections towards the lumen. Arrowheads show nearby V-ATPase-labeled clear cells. Nuclei are visualized with DAPI (blue) (Bars: 5 μm). FIG. 3B shows epididymis stained using anti-AGTR2 antibody with (+ peptide) and without (AGTR2) pre-incubation with the immunizing peptide (Bar: 20 μm). FIG. 3C shows Western blot detection of AGTR2. 180 μg of epididymal homogenates were loaded onto the gel. Two bands at around 44 and 88 kDa were detected (arrows). FIG. 3D shows 3D-reconstruction showing AGTR2-positive basal cells (green; arrows). One basal cell sends a projection between principal cells. Two clear cells, stained apically for the V-ATPase (red), are visible (arrowheads, (Bar: 5 μm). FIG. 3E shows RT-PCR analysis of Agtr2 mRNA expression in clear cells, isolated by FACS from B1-EGFP mouse epididymides (GFP+), and in all other epididymal cell types (GFP−). While a positive signal was detected in the GFP negative cell population, no Agtr2 mRNA expression was observed in GFP-positive clear cells. Lu: lumen. SMC: smooth muscle cells.

FIGS. 4A-4I show luminal ANGII induces V-ATPase apical accumulation in clear cells. FIGS. 4A-4C, show confocal microscopy images of V-ATPase-labeled clear cells (green) luminally perfused in vivo under control conditions (FIG. 4A) or in the presence of 0.1 μM (FIG. 4B) or 1 μM ANGII (FIG. 4C) for 20 min. The arrows show the border between the base of the apical microvilli and the apical pole of the cell (Bars: 5 μm) FIG. 4D shows quantitative analysis of the dose-dependent effect of ANGII on the elongation of V-ATPase-labeled microvilli normalized for the apical width of the cell. Values are mean±SEM obtained from at least 10 cells per epididymis from “n” number of epididymis per group. **P<0.001 vs control. FIGS. 4E and 4F show V-ATPase immunogold labeling of the apical pole of a clear cell perfused under control conditions (FIG. 4E) or in the presence of luminal ANGII (1 μM) (FIG. 4F). Under control conditions, the V-ATPase is located mainly in the sub-apical pole, and a few short V-ATPase-labeled microvilli are detected. In the presence of ANGII, longer and more numerous V-ATPase-labeled microvilli are detected (Bars: 500 nm). FIG. 4G shows apical accumulation of V-ATPase by luminal ANGII (1 μM) in clear cells. The left axis shows the density of V-ATPase-associated gold particles in the apical membrane including microvilli (Gold/μm apical membrane). The right axis shows the total number of gold particles located in the apical membrane of clear cells normalized for the width of the cell (Gold/μm cell width). Twenty eight cells were analyzed in each group. Data are expressed as means±SEM. *P<0.0005. FIG. 4H shows effect of ANGII (1 μM) on proton secretion in cut-open proximal VD using a proton-selective electrode. After an initial spike due to disturbance of the proton gradient, a sustained increase in proton secretion (expressed as μV) was induced by ANGII. A marked inhibition was then observed following addition of concanamycin A (1 μM). FIG. 4I shows mean effect of ANGII (1 μM) on concanamycin-sensitive proton secretion (mean±SEM, n=7) measured 30 min after addition of ANGII. *P<0.05.

FIGS. 5A-5D show AGTR2 mediates ANGII-induced V-ATPase apical accumulation and microvilli elongation in clear cells. FIGS. 5A-5C show confocal images showing clear cells perfused for 20 min with 1 μM ANGII (FIG. 5A), or pre-incubated for 10 min with PD123319 (1 μM; FIG. 5B) or losartan (1 μM; FIG. 5C), before addition of ANGII, still in the presence of antagonists. PD123319, but not losartan, prevented the ANGII-induced microvilli elongation. Arrows show the frontier between the base of apical microvilli and the cytoplasm of the cell. FIG. 5D shows the mean effects of PD123319 and losartan on ANGII-mediated microvilli elongation. PD123319 inhibited the effect of ANGII at both 0.1 and 1 μM concentrations. Values were obtained from at least 10 cells per epididymis. Data are represented as mean±SEM and “n” is the number of rat epididymis examined. *P<0.001 vs control; ns: no significant difference vs control.

FIGS. 6A-6F show NO-sGC-cGMP pathway mediates ANGII-induced V-ATPase apical accumulation and microvilli elongation in clear cells. FIG. 6A shows confocal images of V-ATPase-labeled clear cells (green) perfused under control conditions (left panel), or in the presence of 1 mM p-cpt-cGMP (middle panel), or 1 mM SNP (right panel) for 20 min. A marked elongation of V-ATPase-labelled microvilli is observed in the presence of p-cpt-cGMP and SNP, compared to control. The arrows indicate the border between the base of apical microvilli and the cytoplasm. (Bars: 5 μm). FIG. 6B shows effect of ODQ (middle panel) or L-NAME (right panel) on the ANGII-induced response. Pre-treatment for 10 min with ODQ (3 μM) or L-NAME (100 μM), followed by ANGII still in the presence of inhibitors for 20 min, abolished the V-ATPase apical accumulation and microvilli elongation induced by ANGII alone (Bars: 5 μm). FIG. 6C shows mean microvilli elongation in clear cells. Values were obtained from at least 10 cells per epididymis. Data are represented as mean±SEM, and “n” is the number of epididymides examined. *P<0.01 vs control (CTL), **P<0.001 vs CTL, and #P<0.001 vs ANGII. FIG. 6D shows localization of β₁-sGC (green, upper panel) and V-ATPase (red, middle panel) in epididymis. V-ATPase-positive clear cells (arrows) show abundant β₁-sGC staining in their basolateral membrane and apical pole. Weaker and more uniform staining is also detected in principal, basal, and smooth muscle cells. Nuclei are visualized with DAPI (blue) in the merged panel (lower panel) (Bar: 10 μm). FIG. 6E shows Western blot detection of β₁-sGC in rat (RE) and mouse epididymis (ME) (120 μg/lane). In both samples, a band at about 70 kDa was detected corresponding to the molecular weight of β₁-sGC. Additional bands at around 35 kDa and 50 kDa were also detected in rat and mouse epididymis, respectively, possibly indicating degradation products in these tissues. All bands were absent after pre-incubation of the antibody with the immunizing peptide. FIG. 6F shows inhibition of immunofluorescence staining using the antibody pre-incubated with the immunizing peptide (+peptide) (Bars: 50 μm).

FIG. 7 is a schematic representation of cell to cell cross-talk in the epididymal epithelium. Basal cells extend narrow projections between epithelial cells to reach the lumen. A new TJ is formed between the basal cell and adjacent epithelial cells. Basal cells express AGTR2 and luminal ANGII triggers the production of NO in these cells. NO then acts locally on clear cells to produce cGMP via activation of the sGC, which is enriched in these cells. cGMP induces the accumulation of V-ATPase in well developed apical microvilli in clear cells, which results in the increase of proton secretion.

### DETAILED DESCRIPTION OF THE INVENTION

In one aspect the invention is directed to a method for treating respiratory disorders. The method of treatment comprising inhibiting release of nitric oxide (NO) from a basal cell. Inhibition of nitric oxide release from basal cells can be accomplished in a number of ways, including but not limited to, inhibition of nitric oxide synthesis in the basal cells (e.g., inhibition of nitric oxide synthase), and increased degradation of nitric oxide by NO scavengers in the basal cells.

The term “inhibiting” as used here in relation to nitric oxide release from basal cells means preventing, reducing, or otherwise ameliorating nitric oxide synthesis or its release from basal cells. For example, depending on the circumstances, including nature of the condition being treated, it may not be necessary that inhibition should mean completely blocking of NO synthesis or release from basal cells, but reducing NO synthesis or release to a sufficient degree to enable the desired effect to be achieved.

One embodiment of the invention is a method of treating respiratory disorder comprising the step of administering to a subject a nitric oxide inhibitor. It is to be understood that amount of nitric oxide inhibitor administered is a therapeutically effective amount.

The term “respiratory disorder” refers to any condition and/or disorder relating to respiration and/or the respiratory system. The respiratory disorder can be allergic or non-allergic. In some embodiments, the respiratory disorder is selected from the group consisting of atopic asthma, non-atopic asthma, emphysema, bronchitis, chronic obstructive pulmonary disease (COPD), sinusitis, allergic rhinitis. In some embodiments, the respiratory disorder is characterized by increased responsiveness of the tracheas and bronchi to various stimuli, i.e., allergens, resulting in a widespread narrowing of the airways.

The term “COPD” is generally applied to chronic respiratory disease processes characterized by the persistent obstruction of bronchial air flow. Typical COPD patients are those suffering from conditions such as bronchitis, cystic fibrosis, asthma or emphysema.

In some embodiments, the respiratory disorder is an inflammatory disorder of the lung.

The inventors have discovered that in the male reproductive tract of the rat, basal cells cross the blood/epididymis barrier to monitor luminal factors. Basal cells were the only cells expressing the angiotensin II type 2 receptor, and luminal angiotensin II triggers the production of nitric oxide (NO) in these cells. NO then diffuses out of basal cells and acts locally on neighboring specialized acidifying cells, the so-called clear cells, to produce cGMP via activation of soluble guanylate cyclase, which is enriched in these cells. cGMP induces the accumulation of the proton pumping V-ATPase in the apical membrane of clear cells followed by an increase in proton secretion. The inventors have shown the presence of a novel crosstalk between basal cells and clear cells to control proton secretion by clear cells, a process that is crucial for maintaining sperm quiescent during their maturation and storage in the epididymis. Importantly, numerous basal cells reaching towards the lumen were also observed in the human epididymis, indicating that luminal epididymal sampling by basal cells occurs across species.

This process of luminal sampling by so-called basal cells is a novel mechanism for hormonal signaling that can be generally applicable to other pseudostratified epithelia, including the respiratory tract. Without wishing to be bound by theory, in the upper respiratory tract, the novel property of basal cells to reach the luminal border of the epithelium places these cells in a front-line position to survey foreign pathogenic and allergenic substances that constantly invade this tissue. The basal cells can probe the airway passage for the presence of foreign factors via their luminal projection. They can then communicate with surrounding cells via production of NO, which then act locally in a paracrine manner to stimulate neighboring cells. Dysregulation of this crosstalk pathway may lead to abnormally elevated inflammation of the respiratory tract, leading to diseases such as asthma and COPD. Therefore, targeting nitric oxide release from basal cells specifically spares the other functions of the lung that depend on the NO pathway, including its vasodilatory action of the pulmonary blood vessels.

### Nitric Oxide Inhibitors

As used herein, the term “nitric oxide inhibitor” refers to any composition or agent that inhibits the production of nitric oxide or scavenges or removes existing nitric oxide. A nitric oxide inhibitor can be a small molecule, peptide, peptide mimetic, protein or a portion thereof, antibody, nucleic acid (e.g. antisense oligonucleotide, siRNA, miRNA, miRNA mimic, antagomir, ribozyme, aptamer, and decoy oligonucleotide), or gene therapy reagent.

As used herein, the term “siRNA” refers double stranded nucleic acids and which double stranded nucleic acids have the ability to reduce or inhibit expression of a gene or target gene in a cell when the siRNA is introduced into the cells. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 or about 24-29 nucleotides in length, e.g., 20, 21, 22, 23, 24, 25, 26, 27, 28, 29, or 30 nucleotides in length.

An “antibody” that can be used according to the methods described herein includes complete immunoglobulins, antigen binding fragments of immunoglobulins, as well as antigen binding proteins that comprise antigen binding domains of immunoglobulins. Antigen binding fragments of immunoglobulins include, for example, Fab, Fab′, F(ab′)₂, scFv and dAbs. Modified antibody formats have been developed which retain binding specificity, but have other characteristics that may be desirable, including for example, bispecificity, multivalence (more than two binding sites), and compact size (e.g., binding domains alone). Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies. Multiple single chain antibodies, each single chain having one VH and one VL domain covalently linked by a first peptide linker, can be covalently linked by at least one or more peptide linker to form multivalent single chain antibodies, which can be monospecific or multispecific. Each chain of a multivalent single chain antibody includes a variable light chain fragment and a variable heavy chain fragment, and is linked by a peptide linker to at least one other chain. The peptide linker is composed of at least fifteen amino acid residues. The maximum number of linker amino acid residues is approximately one hundred. Two single chain antibodies can be combined to form a diabody, also known as a bivalent dimer. Diabodies have two chains and two binding sites, and can be monospecific or bispecific. Each chain of the diabody includes a VH domain connected to a VL domain. The domains are connected with linkers that are short enough to prevent pairing between domains on the same chain, thus driving the pairing between complementary domains on different chains to recreate the two antigen-binding sites. Three single chain antibodies can be combined to form triabodies, also known as trivalent trimers. Triabodies are constructed with the amino acid terminus of a VL or VH domain directly fused to the carboxyl terminus of a VL or VH domain, i.e., without any linker sequence. The triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific. Thus, antibodies useful in the methods described herein include, but are not limited to, naturally occurring antibodies, bivalent fragments such as (Fab′)₂, monovalent fragments such as Fab, single chain antibodies, single chain Fv (scFv), single domain antibodies, multivalent single chain antibodies, diabodies, triabodies, and the like that bind specifically with an antigen.

Antibodies can also be raised against a polypeptide or portion of a polypeptide by methods known to those skilled in the art. Antibodies are readily raised in animals such as rabbits or mice by immunization with the gene product, or a fragment thereof. Immunized mice are particularly useful for providing sources of B cells for the manufacture of hybridomas, which in turn are cultured to produce large quantities of monoclonal antibodies. Antibody manufacture methods are described in detail, for example, in Harlow et al., 1988 in: Antibodies, A Laboratory Manual, Cold Spring Harbor, N.Y. While both polyclonal and monoclonal antibodies can be used in the methods described herein, it is preferred that a monoclonal antibody is used where conditions require increased specificity for a particular protein. In some embodiments, nitric oxide inhibitor is an antibody against an extracellular epitope of the basal cells

In some embodiments, the nitric oxide inhibitor inhibits the endothelial nitric oxide, e.g., by inhibiting the expression of endothelial nitric oxide synthase.

Exemplary NO inhibitors include, but are not limited to, 2-phenyl-4,4,5,5tetraethylimidazoline-1-oxyl-3-oxide (PTIO), 2-(4carboxyphenyl)-4,4,5,5-tetraethylimidazoline-1-oxyl-3oxide (Carboxy-PTIO), N-methyl-D-glucamine dithiocarbamate (MOD), N-nitro-L-arginine methyl-ester (L-NAME), N-monomethyl-L-arginine (L-NMMA), 2-ethyl-2-thiopseudourea (ETU), 2-methylisothiourea (SMT), 7-nitroindazole, L-arginine analogues (such as aminoguanidine, aminoguanidine hemisulfate, N-monomethyl-L-arginine, N-nitro-L-arginine, D-arginine and the like) diphenyleneiodonium (DPI), L-cysteine, heparin, SC-51, Vitamin B12 (hydroxocobalamin), cyanocobalamin, hemoglobin, flavohemoglobin, heme, myoglobin, ruthenium (III) polyaminocarboxylate complex, NOX-100, NOX-200, NOX-700, dithiocarbamates which chelate iron to form complex which irreversibly binds free NO including pyrrolidine dithiocarbamate, and esters and prodrugs thereof.

Other nitric oxide inhibitors amenable to the invention are described in U.S. Pat. Nos. 5,216,025; 5,585,402; 5,830,917; 6,545,170; 5,273,875; 5,266,594; 5,629,322; 5,918,787, 5,981,511; 5,929,085; 5,945,408; 5,972,975; 5,854,234; 5,863,931; 5,908,842; 6,545,170; 5,132,453; 5,674,907; 6403,830; 6,344,483; 5,929,063; 6,274,55; 5,723,451; 6,465,686; 6,586,474; 6,485,544; 6,756,406; and 6,432,947, contents of all of which are herein incorporated by reference in their entirety.

Nitric oxide inhibitor can be linked with a ligand. Without wishing to be bound by theory, the ligand can target the nitric oxide inhibitor to basal cells and/or enhance uptake of the nitric oxide inhibitors by basal cells. In some embodiments, the ligand is an antibody specific for an extracellular epitope of basal cells. In some embodiments, the ligand is a basal specific lectin.

### Pharmaceutical Compositions

For administration to a subject, the nitric oxide inhibitors can be provided in pharmaceutically acceptable compositions The pharmaceutically acceptable compositions comprise a therapeutically-effective amount of one or more NO inhibitors, formulated together with one or more pharmaceutically acceptable carriers (additives) and/or diluents. As described in detail below, the pharmaceutical compositions of the present invention can be specially formulated for administration in solid, liquid or gel form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally HIF inhibitors can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquids such as suspensions (e.g., aqueous or non-aqueous liquid suspensions, oil-in-water emulsions, or water-in-oil liquid emulsions), solutions, and elixirs; and sterile solids (e.g., crystalline or amorphous solids) that can be reconstituted to provide liquid dosage forms.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of HIF inhibitor present in the pharmaceutical composition.

Additionally, the NO inhibitors can be delivered using lipid- or polymer-based nanoparticles. See for example Allen, T. M., Cullis, P. R. Drug delivery systems: entering the mainstream. Science. 303(5665): 1818-22 (2004).

The amount of NO inhibitor which can be combined with a carrier material to produce a single dosage form will generally be that amount of the NO inhibitor which produces a therapeutic effect. Generally out of one hundred percent, this amount will range from about 0.01% to 99% of NO inhibitor, preferably from about 0.1% to about 70%, most preferably from 5% to about 30%.

The phrase “therapeutically-effective amount” as used herein means that amount of a compound, material, or composition which is effective for producing some desired therapeutic effect in at least a sub-population of cells in an animal at a reasonable benefit/risk ratio applicable to any medical treatment. For example, an amount of a compound administered to a subject that is sufficient to produce a statistically significant, measurable change in at least one symptom of respiratory disorder. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, the term “administer” refers to the placement of a NO inhibitor into a subject by a method or route which results in at least partial localization of the NO inhibitor at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. The term “treatment” of a disease also includes providing relief from the symptoms or side-effects of the disease (including palliative treatment). For example, any reduction in inflammation, bronchospasm, bronchoconstriction, shortness of breath, wheezing, lower extremity edema, ascites, productive cough, hemoptysis, or cyanosis in subject suffering from a respiratory disorder, no matter how slight, would be considered an alleviated symptom. In one embodiment, the symptoms of a disease or disorder are alleviated by at least 5%, at least 10%, at least 20%, at least 30%, at least 40%, or at least 50%.

In methods of treatment described herein, the administration of NO inhibitor can be for either “prophylactic” or “therapeutic” purpose. When provided prophylactically, the NO inhibitor is provided in advance of any symptom. The prophylactic administration of the NO inhibitor serves to prevent or inhibit any nitric oxide release at a site. When provided therapeutically, the NO inhibitor is provided at (or after) the onset of a symptom or indication of respiratory disorder. Thus, NO inhibitor can be provided prior to the onset of respiratory disorder, e.g., on set of an allergic respiratory disorder.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices, are preferred.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

The dosage may be determined by a physician and adjusted, as necessary, to suit observed effects of the treatment. Generally, the compositions are administered so that NO inhibitor is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, 10 mg/kg to 20 mg/kg, or 0.01 pg/kg to 1 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg etc. It is to be further understood that the ranges intermediate to the given above are also within the scope of this invention, for example, the range 1 mg/kg to 10 mg/kg includes dose ranges such as 2 mg/kg to 8 mg/kg, 3 mg/kg to 7 mg/kg, 4 mg/kg to 6 mg/kg etc.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

### Aerosol Formulations

NO inhibitors can be administered directly to the airways in the form of an aerosol or by nebulization. For use as aerosols, NO inhibitors in solution or suspension may be packaged in a pressurized aerosol container together with suitable propellants, for example, hydrocarbon propellants like propane, butane, or isobutane with conventional adjuvants. The NO inhibitors can also be administered in a non-pressurized form such as in a nebulizer or atomizer.

The term “nebulization” is well known in the art to include reducing liquid to a fine spray. Preferably, by such nebulization small liquid droplets of uniform size are produced from a larger body of liquid in a controlled manner. Nebulization can be achieved by any suitable means therefor, including by using many nebulizers known and marketed today. For example, an AEROMIST pneumatic nebulizer available from Inhalation Plastic, Inc. of Niles, Ill.

As is well known, any suitable gas can be used to apply pressure during the nebulization, with preferred gases to date being those which are chemically inert to the NO inhibitors. Exemplary gases including, but are not limited to, nitrogen, argon or helium can be used to high advantage.

NO inhibitors can also be administered directly to the airways in the form of a dry powder. For use as a dry powder, NO inhibitors can be administered by use of an inhaler. Exemplary inhalers include metered dose inhalers and dry powdered inhalers. A metered dose inhaler or “MDI” is a pressure resistant canister or container filled with a product such as a pharmaceutical composition dissolved in a liquefied propellant or micronized particles suspended in a liquefied propellant. The correct dosage of the composition is delivered to the patient. A dry powder inhaler is a system operable with a source of pressurized air to produce dry powder particles of a pharmaceutical composition that is compacted into a very small volume.

Dry powder aerosols for inhalation therapy are generally produced with mean diameters primarily in the range of <5 μm. As the diameter of particles exceeds 3 μm, there is increasingly less phagocytosis by macrophages. However, increasing the particle size also has been found to minimize the probability of particles (possessing standard mass density) entering the airways and acini due to excessive deposition in the oropharyngeal or nasal regions.

Suitable powder compositions include, by way of illustration, powdered preparations of NO inhibitors thoroughly intermixed with lactose, or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants, and co-solvents and may be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic an diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

### Oral Dosage Forms

Pharmaceutical NO inhibitor compositions of the disclosure that are suitable for oral administration can be presented as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington's Pharmaceutical Sciences, 18th ed., Mack Publishing, Easton, Pa. (1990).

Typical oral dosage forms of the compositions of the disclosure are prepared by combining the pharmaceutically acceptable salt of disclosed compounds in an intimate admixture with at least one excipient according to conventional pharmaceutical compounding techniques. Excipients can take a wide variety of forms depending on the form of the composition desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, kaolin, diluents, granulating agents, lubricants, binders, and disintegrating agents. Due to their ease of administration, tablets and capsules represent the most advantageous solid oral dosage unit forms, in which case solid pharmaceutical excipients are used. If desired, tablets can be coated by standard aqueous or nonaqueous techniques. These dosage forms can be prepared by any of the methods of pharmacy. In general, pharmaceutical compositions and dosage forms are prepared by uniformly and intimately admixing the active ingredient(s) with liquid carriers, finely divided solid carriers, or both, and then shaping the product into the desired presentation if necessary.

For example, a tablet can be prepared by compression or molding. Compressed tablets can be prepared by compressing in a suitable machine the active ingredient(s) in a free-flowing form, such as a powder or granules, optionally mixed with one or more excipients. Molded tablets can be made by molding in a suitable machine a mixture of the powdered compound moistened with an inert liquid diluent. Examples of excipients that can be used in oral dosage forms of the disclosure include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, and AVICEL-PH-105 (available from FMC Corporation, American Viscose Division, Avicel Sales, Marcus Hook, Pa., U.S.A.), and mixtures thereof. An exemplary suitable binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the disclosure is typically present in from about 50 to about 99 weight percent of the pharmaceutical composition or dosage form.

Disintegrants are used in the compositions of the disclosure to provide tablets that disintegrate when exposed to an aqueous environment. Tablets that contain too much disintegrant may swell, crack, or disintegrate in storage, while those that contain too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) should be used to form solid oral dosage forms of the disclosure. The amount of disintegrant used varies based upon the type of formulation and mode of administration, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used to form pharmaceutical compositions and dosage forms of the disclosure include, but are not limited to, agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, and mixtures thereof.

Lubricants that can be used to form pharmaceutical compositions and dosage forms of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL® 200, manufactured by W. R. Grace Co. of Baltimore, Md.), a coagulated aerosol of synthetic silica (marketed by Degussa Co. of Piano, Tex.), CAB-O-SIL® (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

This disclosure further encompasses lactose-free pharmaceutical compositions and dosage forms, wherein such compositions preferably contain little, if any, lactose or other mono- or di-saccharides. As used herein, the term “lactose-free” means that the amount of lactose present, if any, is insufficient to substantially increase the degradation rate of an active ingredient.

Lactose-free compositions of the disclosure can comprise excipients which are well known in the art and are listed in the USP (XXI)/NF (XVI), which is incorporated herein by reference. In general, lactose-free compositions comprise a pharmaceutically acceptable salt of an HIF inhibitor, a binder/filler, and a lubricant in pharmaceutically compatible and pharmaceutically acceptable amounts. Preferred lactose-free dosage forms comprise a pharmaceutically acceptable salt of the disclosed compounds, microcrystalline cellulose, pre-gelatinized starch, and magnesium stearate.

This disclosure further encompasses anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

For oral administration, the dosage should contain at least at least 0.1% of HIF inhibitor. The percentage of HIF inhibitor in these compositions may, of course, be varied and may conveniently be between about 2% to about 60% of the weight of the unit. The amount of HIF inhibitor in such therapeutically useful compositions is such that a suitable dosage will be obtained. Preferred compositions according to the present invention are prepared so that an oral dosage unit contains between about 1 and 250 mg of HIF inhibitor.

### Controlled and Delayed Release Dosage Forms

Nitric oxide inhibitors can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, Duolite® A568 and Duolite® AP143 (Rohm&Haas, Spring House, Pa. USA).

One embodiment of the disclosure encompasses a unit dosage form that includes a pharmaceutically acceptable salt of the disclosed compounds (e.g., a sodium, potassium, or lithium salt), or a polymorph, solvate, hydrate, dehydrate, co-crystal, anhydrous, or amorphous form thereof, and one or more pharmaceutically acceptable excipients or diluents, wherein the pharmaceutical composition or dosage form is formulated for controlled-release. Specific dosage forms utilize an osmotic drug delivery system.

A particular and well-known osmotic drug delivery system is referred to as OROS® (Alza Corporation, Mountain View, Calif. USA). This technology can readily be adapted for the delivery of compounds and compositions of the disclosure. Various aspects of the technology are disclosed in U.S. Pat. Nos. 6,375,978 B1; 6,368,626 B1; 6,342,249 B1; 6,333,050 B2; 6,287,295 B1; 6,283,953 B1; 6,270,787 B1; 6,245,357 B1; and 6,132,420; each of which is incorporated herein by reference. Specific adaptations of OROS® that can be used to administer compounds and compositions of the disclosure include, but are not limited to, the OROS® Push-Pull™, Delayed Push-Pull™, Multi-Layer Push-Pull™, and Push-Stick™ Systems, all of which are well known. See, e.g. worldwide website alza.com. Additional OROS® systems that can be used for the controlled oral delivery of compounds and compositions of the disclosure include OROS®-CT and L-OROS®; see, Delivery Times, vol. 11, issue II (Alza Corporation).

Conventional OROS® oral dosage forms are made by compressing a drug powder (e.g., a HIF inhibitor salt) into a hard tablet, coating the tablet with cellulose derivatives to form a semi-permeable membrane, and then drilling an orifice in the coating (e.g., with a laser). Kim, Cherng-ju, Controlled Release Dosage Form Design, 231-238 (Technomic Publishing, Lancaster, Pa.: 2000). The advantage of such dosage forms is that the delivery rate of the drug is not influenced by physiological or experimental conditions. Even a drug with a pH-dependent solubility can be delivered at a constant rate regardless of the pH of the delivery medium. But because these advantages are provided by a build-up of osmotic pressure within the dosage form after administration, conventional OROS® drug delivery systems cannot be used to effectively delivery drugs with low water solubility.

A specific dosage form of the NO inhibitor compositions of the disclosure includes: a wall defining a cavity, the wall having an exit orifice formed or formable therein and at least a portion of the wall being semipermeable; an expandable layer located within the cavity remote from the exit orifice and in fluid communication with the semipermeable portion of the wall; a dry or substantially dry state drug layer located within the cavity adjacent the exit orifice and in direct or indirect contacting relationship with the expandable layer; and a flow-promoting layer interposed between the inner surface of the wall and at least the external surface of the drug layer located within the cavity, wherein the drug layer includes a NO inhibitor, or a polymorph, solvate, hydrate, dehydrate, co-crystal, anhydrous, or amorphous form thereof. See U.S. Pat. No. 6,368,626, the entirety of which is incorporated herein by reference.

Another specific dosage form of the disclosure includes: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a liquid, active agent formulation absorbed in porous particles, the porous particles being adapted to resist compaction forces sufficient to form a compacted drug layer without significant exudation of the liquid, active agent formulation, the dosage form optionally having a placebo layer between the exit orifice and the drug layer, wherein the active agent formulation comprises a NO inhibitor, or a polymorph, solvate, hydrate, dehydrate, co-crystal, anhydrous, or amorphous form thereof. See U.S. Pat. No. 6,342,249, the entirety of which is incorporated herein by reference.

In some embodiments, the NO inhibitory compound is administered to a subject by sustained release or in pulses. Pulse therapy is not a form of discontinuous administration of the same amount of a composition over time, but comprises administration of the same dose of the composition at a reduced frequency or administration of reduced doses. Sustained release or pulse administration are particularly preferred when the angiogenesis is associated with tumor growth as it leads to regression of blood vessels feeding the tumor and ultimately to regression of the tumor itself. Each pulse dose can be reduced and the total amount of drug administered over the course of treatment to the patient is minimized.

Individual pulses can be delivered to the patient continuously over a period of several hours, such as about 2, 4, 6, 8, 10, 12, 14 or 16 hours, or several days, such as 2, 3, 4, 5, 6, or 7 days, preferably from about 1 hour to about 24 hours and more preferably from about 3 hours to about 9 hours.

The interval between pulses or the interval of no delivery is greater than 24 hours and preferably greater than 48 hours, and can be for even longer such as for 3, 4, 5, 6, 7, 8, 9 or 10 days, two, three or four weeks or even longer. As the results achieved may be surprising, the interval between pulses, when necessary, can be determined by one of ordinary skill in the art. Often, the interval between pulses can be calculated by administering another dose of the composition when the composition or the active component of the composition is no longer detectable in the patient prior to delivery of the next pulse. Intervals can also be calculated from the in vivo half-life of the composition. Intervals may be calculated as greater than the in vivo half-life, or 2, 3, 4, 5 and even 10 times greater the composition half-life.

The number of pulses in a single therapeutic regimen may be as little as two, but is typically from about 5 to 10, 10 to 20, 15 to 30 or more. In fact, patients can receive drugs for life according to the methods of this invention without the problems and inconveniences associated with current therapies. Compositions can be administered by most any means, but are preferable delivered to the patient as an injection (e.g. intravenous, subcutaneous, intraarterial), infusion or instillation. Various methods and apparatus for pulsing compositions by infusion or other forms of delivery to the patient are disclosed in U.S. Pat. Nos. 4,747,825; 4,723,958; 4,948,592; 4,965,251 and 5,403,590.

In a certain embodiments, the interval between pulses is 24 hours or greater.

In a certain embodiments, the plurality of pulses comprises from about 5 to about 10 pulses.

In a certain embodiments, the plurality of pulses comprises greater than 20 pulses.

In a certain embodiments, the interval is from 1 to about 7 days.

Sustained release may be accomplished by means of an osmotic pump. In some embodiments NO inhibitor is administered over a period of several days, such as 2, 3, 4, 5, 6 or 7 days.

### Parenteral Dosage Forms

Parenteral dosage forms can be administered to patients by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, administration DUROS®-type dosage forms, and dose-dumping.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Compounds that alter or modify the solubility of a pharmaceutically acceptable salt of a HIF inhibitor disclosed herein can also be incorporated into the parenteral dosage forms of the disclosure, including conventional and controlled-release parenteral dosage forms.

### Topical, Transdermal and Mucosal Dosage Forms

Topical dosage forms of the disclosure include, but are not limited to, creams, lotions, ointments, gels, shampoos, sprays, aerosols, solutions, emulsions, and other forms known to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 18.sup.th Ed., Mack Publishing, Easton, Pa. (1990).

Transdermal and mucosal dosage forms of the NO inhibitor compositions of the disclosure include, but are not limited to, ophthalmic solutions, patches, sprays, aerosols, creams, lotions, suppositories, ointments, gels, solutions, emulsions, suspensions, or other forms known to one of skill in the art. See, e.g., Remington's Pharmaceutical Sciences, 18th Ed., Mack Publishing, Easton, Pa. (1990); and Introduction to Pharmaceutical Dosage Forms, 4th Ed., Lea & Febiger, Philadelphia, Pa. (1985). Dosage forms suitable for treating mucosal tissues within the oral cavity can be formulated as mouthwashes, as oral gels, or as buccal patches. Additional transdermal dosage forms include “reservoir type” or “matrix type” patches, which can be applied to the skin and worn for a specific period of time to permit the penetration of a desired amount of active ingredient.

Examples of transdermal dosage forms and methods of administration that can be used to administer the active ingredient(s) of the disclosure include, but are not limited to, those disclosed in U.S. Pat. Nos. 4,624,665; 4,655,767; 4,687,481; 4,797,284; 4,810,499; 4,834,978; 4,877,618; 4,880,633; 4,917,895; 4,927,687; 4,956,171; 5,035,894; 5,091,186; 5,163,899; 5,232,702; 5,234,690; 5,273,755; 5,273,756; 5,308,625; 5,356,632; 5,358,715; 5,372,579; 5,421,816; 5,466;465; 5,494,680; 5,505,958; 5,554,381; 5,560,922; 5,585,111; 5,656,285; 5,667,798; 5,698,217; 5,741,511; 5,747,783; 5,770,219; 5,814,599; 5,817,332; 5,833,647; 5,879,322; and 5,906,830, each of which are incorporated herein by reference in their entirety.

Suitable excipients (e.g., carriers and diluents) and other materials that can be used to provide transdermal and mucosal dosage forms encompassed by this disclosure are well known to those skilled in the pharmaceutical arts, and depend on the particular tissue or organ to which a given pharmaceutical composition or dosage form will be applied. With that fact in mind, typical excipients include, but are not limited to water, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable.

Depending on the specific tissue to be treated, additional components may be used prior to, in conjunction with, or subsequent to treatment with an HIF inhibitor of the disclosure. For example, penetration enhancers can be used to assist in delivering the active ingredients to or across the tissue. Suitable penetration enhancers include, but are not limited to: acetone; various alcohols such as ethanol, oleyl, an tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate).

The pH of a pharmaceutical composition or dosage form, or of the tissue to which the pharmaceutical composition or dosage form is applied, may also be adjusted to improve delivery of the active ingredient(s). Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of the active ingredient(s) so as to improve delivery. In this regard, stearates can serve as a lipid vehicle for the formulation, as an emulsifying agent or surfactant, and as a delivery-enhancing or penetration-enhancing agent. Different hydrates, dehydrates, co-crystals, solvates, polymorphs, anhydrous, or amorphous forms of the pharmaceutically acceptable salt of an HIF inhibitor can be used to further adjust the properties of the resulting composition.

### Combination Treatments

Nitric oxide inhibitors can be administrated to a subject in combination with a pharmaceutically active agent. Exemplary pharmaceutically active compound include, but are not limited to, those found in Harrison's Principles of Internal Medicine, 13^{th} Edition, Eds. T. R. Harrison et al. McGraw-Hill N.Y., N.Y.; Physicians Desk Reference, 50^{th} Edition, 1997, Oradell N.J., Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete contents of all of which are incorporated herein by reference. Without wishing to be bound by theory, administration of nitric oxide in combination with a pharmaceutically active agent can increase the effectiveness of nitric oxide.

In some embodiments, the pharmaceutically active agent is an asthma agent. Exemplary agents known to treat asthma include mast cell degranulation agents (i.e., Cromylyn sodium or Nedocromil sodium), leukotriene inhibitors (i.e., Monteleukast sodium, Zafirlukast, or Pranlukast hydrate), corticosteroids (i.e., Beclomethasone, Budesonide, Ciclesonide, Hydrolysable glucocorticoid, Triamcinolone acetonide, Flunisolide, Mometasone furcate, or Fluticasone propionate), (3-Antagonists (i.e., Albuterol, Bambuterol, Formoterol, Salbutamol, Terbutaline sulfate, or Salmeterol), IgE binding inhibitors (i.e., Omalizumab), Adenosine A2 agonists, Anti-CD23 antibody, E-Selectin antagonists, P-Selectin antagonists, L-Selectin antagonists, interlukin inhibitors/monoclonal antibodies, pulmonary surfactants, neurokinin antagonists, NF-Kappa-B inhibitors, PDE-4 inhibitors (i.e., Cilomilast, or Roflumilast), Thromboxan A2 inhibitors (i.e., Rama-go troban, or Seratrodast), tryptase inhibitors, VIP agonists or antisense agents.

In some embodiments, the pharmaceutically active agent is an agent used to treat allergic rhinitis. Exemplary agents include, but are not limited to HI antihistamines i.e., terfendine or astemizole; alpha-adrenergic agents; and glucocorticoids, i.e., beclamethasone or flunisolide.

In some embodiments, the pharmaceutically active agent is an agent to treat sinusitis, more specifically, chronic sinusitis. Exemplary agents to treat sinusitis include, but are not limited to, corticosteroids (e.g., oral, intranasal, nebulized, or inhaled); antibiotics (e.g., oral, intranasal, nebulized, inhaled or intravenous); anti-fungal agents; salt-water nasal washes and mist sprays; anti-inflammatory agents; decongestants (oral or nasal); guaifenesin; potassium iodide; leukotriene inhibitors (e.g., monteleukast); mast cell degranulating agents; topical moisterizing applications (e.g., nasal sprays or gels which may contain moisterizing agents such as propylene glycol or glycerin); hot air inhalation; mechanical devices to aid in breathing; enzymatic cleansers (e.g., papaya enzymes); and antihistamine sprays.

In some embodiments, the pharmaceutically active agent is an agent to treat COPD or chronic bronchitis or emphysema. Exemplary agents to treat COPD include, but are not limited to, bronchodilator drugs [e.g., short and long acting beta-2 stimulants, anticholinergics (e.g., ipratoprium bromide, theophylline compounds or a combination), steroids (topical or oral), or mucolytic agents (e.g., ambroxol, ergosterin, carbocysteine, iodinated glycerol)]; antibiotics; anti-fungals; moisterization by nebulization; anti-tussives; respiratory stimulants (e.g., doxapram, almitrine bismesylate); and alpha 1 antitrypsin administration.

Nitric oxide inhibitor and pharmaceutically active agent can be administrated to the subject in the same pharmaceutical composition or in different pharmaceutical compositions (at the same time or at different times). When administrated at different times, compound of the invention and the pharmaceutically active agent can be administered within 5 minutes, 10 minutes, 20 minutes, 60 minutes, 2 hours, 3 hours, 4, hours, 8 hours, 12 hours, 24 hours of administration of the other. When NO inhibitor and the pharmaceutically active agent are administered in different pharmaceutical compositions, routes of administration can be different. For example, an NO inhibitor can be administered by any appropriate route known in the art including, but not limited to oral or parenteral routes, including intravenous, intramuscular, subcutaneous, transdermal, airway (aerosol), pulmonary, nasal, rectal, and topical (including buccal and sublingual) administration, and pharmaceutically active agent is administration by a different route, e.g. a route commonly used in the art for administration of said pharmaceutically active agent.

The NO inhibitor may precede, be co-current with and/or follow the pharmaceutically active agent by intervals ranging from minutes to weeks. In embodiments where the NO inhibitor and pharmaceutically active agent are applied separately to a cell, tissue or organism, one would generally ensure that a significant period of time did not expire between the time of each delivery, such that the NO inhibitor and pharmaceutically active agent would still be able to exert an advantageously combined effect on the cell, tissue or organism.

In some embodiments, the invention contemplates the practice of the methods described herein in conjunction with other therapies such as surgery, e.g., enlarging a sinus passage, remove obstructing bone or nasal polyps, mucosal stripping, removal of sinuses, bullectomy, lung volume reduction surgery, or lung transplantation.

### Discussion

The inventors have discovered that narrow projections emanating from so-called basal cells can actually reach the luminal side of an epithelium. This previously unrecognized property of basal cells was observed in several tissues located in the male reproductive tract and upper respiratory tract, indicating that it is a widespread phenomenon that could have general significance to the biology of pseudostratified epithelia.

In the trachea, two types of basal cells have been described: the so-called basal cells, which appear to nestle beneath columnar epithelial cells, and tall basal cells, which extend processes between other epithelial cells (Evans et al., 2001). The property of basal cells to reach the luminal border of the epithelium places these cells in a position to survey foreign pathogenic and allergenic substances that constantly invade the upper respiratory tract.

Two distinct tissues of the male reproductive tract have basal cells that contact the luminal milieu: the coagulating gland and the epididymis. Interestingly, the coagulating gland in rodents is analogous to the middle lobe of the human prostate (Price, 1963; Wei et al., 1997). The notion that basal cells can reach the prostate lumen has significant impact in understanding of the (patho)physiology of this important organ. Numerous basal cells reaching towards the lumen were also observed in the rat and human epididymis, indicating that luminal epididymal sampling by basal cells occurs across species.

In the epididymis, the inventors discovered that basal cells can cross the blood/epididymis barrier while preserving its integrity. The basal cells do so by establishing a new TJ between themselves and adjacent epithelial cells. While sending their body projections towards the lumen, basal cells often seem to “stop short” just beneath the TJs of the epithelium. While virtually no such cells were detected in the proximal regions, the number of basal cells reaching the luminal border dramatically increased in the distal corpus and proximal cauda. This indicates that the capacity of basal cells to reach the lumen is a dynamic property that is locally regulated in different regions of the epididymis. A fraction of the total number of basal cells could be seen crossing the TJs at one given time in still images, indicating a potential dynamic interplay between these cells and the epithelium. Thus, basal cells can “come and go” to and from the lumen, and the establishment of a new TJ between basal cells and epithelial cells, in addition to being dynamic, can be temporary. The time required for a leukocyte to cross the TJ barrier of endothelia is less than 2 min (Stein et al., 1997), and it is conceivable that basal cells can modulate the epididymal barrier in a shorter time frame. The signal responsible for inducing basal cells to interact with and cross the TJ barrier remains unclear.

Interestingly, basal cells always reached TJs at the regions where three epithelial cells intersect (tricellular corners), a feature also described for neutrophils crossing endothelial barriers (Burns et al., 2000). Most remarkably, basal cells can actually open up and cross these TJs. The continuous ZO1 labeling in the region of contact between these cells and adjacent epithelial cells suggests that new TJs had been established. High expression of Cldn1 in epididymal basal cells (as described herein and (Gregory et al., 2001)), as well as in principal cells, at lower levels (Gregory et al., 2001) can provide a molecular “grip” by which basal cells extend projections towards the lumen. Cldn1 forms pairs not only with itself, but also with other claudins, including Cldn3 and Cldn4 (Schneeberger and Lynch, 2004), which are expressed in epididymal TJs (Gregory and Cyr, 2006). This can contribute to the formation of a new TJ between the penetrating basal cell and adjacent epithelial cells. TJ strands constantly form and reform, without disturbing their barrier function (Schneeberger and Lynch, 2004). This remodeling allows migration of leukocytes across endothelia (Burns et al., 2000), as well as penetration of dendritic cells, which also express Cldn1, across the intestinal epithelium (Niess et al., 2005; Rescigno et al., 2001) and the upper respiratory tract (Takano et al., 2005).

The inventors further discovered that activation of AGTR2 by luminal ANGII stimulates proton secretion by epididymal clear cells via activation of the NO/cGMP pathway. NO is a downstream effector of AGTR2 and because basal cells are the only cell type in which this receptor is expressed, they are the likely site for ANGII-induced NO production. A schematic view of a cell-cell cross-talk model for activation of proton secretion in clear cells following AGTR2 stimulation in basal cells is illustrated in FIG. 7. Consistent with this model, endothelial NO synthases (eNOS) have been detected in basal-like cells in human and bovine epididymis (Mewe et al., 2006; Zini et al., 1996). Sampling of luminal ANGII by basal cells followed by activation of proton secretion by clear cells can ensure that the luminal fluid is maintained at its physiological acidic pH. Cross-talk between basal and principal cells has also been proposed to modulate anion secretion by principal cells in response to basolateral lysylbradykinin (Cheung et al., 2005; Leung et al., 2004).

Basolateral stimulation of AGTR1 by ANGII activates anion secretion in cultured principal cells (Leung et al., 1997; Leung and Sernia, 2003), and inventor have shown that the epididymis can respond to luminal ANGII. In agreement with this observation, recent reports showed that ANGII increases V-ATPase-dependent proton extrusion by renal intercalated cells (Pech et al., 2008; Rothenberger et al., 2007), which are analogous to clear cells (Breton and Brown, 2007). Inventors had previously shown that cAMP elevation following activation of the bicarbonate sensitive soluble adenylyl cyclase (sAC) and PKA, induced apical accumulation of the V-ATPase into well-developed microvilli in clear cells (Pastor-Soler et al., 2003; Pastor-Soler et al., 2008). The inventors have also discovered that cGMP can also activate V-ATPase-dependent luminal acidification.

Spermatozoa require an acidic environment to prevent their premature activation during maturation and storage in the epididymis (Hinton and Palladino, 1995; Jones and Murdoch, 1996; Pastor-Soler et al., 2005). However, the mechanisms by which spermatozoa interact with epithelial cells of the epididymal tubule remain, for the most part, unknown. In ACE KO mice, absence of the germinal form of ACE (gACE) induces a marked reduction in the quality of sperm, which are unable to fertilize an egg (Esther et al., 1996; Hagaman et al., 1998; Krege et al., 1995). gACE, which is linked to the sperm membrane (Kondoh et al., 2005), is shed from the sperm surface as they mature in the epididymis (Gatti et al., 1999), providing a potential means by which spermatozoa communicate with surrounding epithelial cells. The lack of luminal ANGII in ACE KO male mice can impair the acidifying capacity of the epididymis with detrimental consequences on sperm quality. Indeed, FOXI-1 KO male mice, which have impaired luminal acidification, are also infertile due to sperm inability to fertilize an egg (Blomqvist et al., 2006).

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, ““reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”,“increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

The term “basal cell” is a general term applied to any stratified or pseudostratified epithelium. It refers to cells which are juxtaposed to the basement membrane and under one or more additional epithelial layers. Many tissue can have both a two cell layer epithelium (basal and luminal cells) or a single layered epithelium. In the two cell layer, the cells adjacent to the basement membrane are termed “basal cells.”

The term “epitope” as used herein refers to that portion of an antigen that makes contact with a particular antibody.

As used herein, the term “basal cell epitope” refers to an epitope that is present on the surface of the basal cell and whose expression is characteristic of basal cells.

The term “asthma” as used herein is defined as a disease of the airways that is characterized by increased responsiveness of the tracheobronchial tree to a multiplicity of stimuli.

The term “allergic respiratory disorder” or “hypersensitivity disease” refers to allergic diseases and/or disorders of the lungs or respiratory system. Allergic disorders are characterized by hypersensitivity to an allergen.

The term “atopic” as used herein refers to a state of atopy or allergy to an allergen or a state of hypersensitivity to an allergen. Typically, atopic refers to Type I hypersensitivity which results from release of mediators (e.g., histamine and/or leukotrines) from IgE-sensitized basophils and mast cells after contact with an antigen (allergen). An example of atopic is atopic asthma, which is allergic asthma and is characterized by an IgE response.

The term “allergen” as used herein refers to an innocuous antigen that induces an allergic or hypersensitive reaction.

The term “allergic rhinitis” as used herein is characterized by any of the following symptoms: obstruction of the nasal passages, conjuctival, nasal and pharyngeal itching, lacrimation, sneezing, or rhinorrhea. These symptoms usually occur in relationship to allergen exposure.

The term “non-allegic” as used herein refers to a respiratory disorder that is not a result from or caused by an allergen. Thus, the non-allergic respiratory disorder is 55 caused by other mechanisms not relating to hypersensitivity to air inocuous agent or allergen.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used as subjects that represent animal models of respiratory disorders. In addition, the methods described herein can be used to treat domesticated animals and/or pets. A subject can be male or female. A subject can be one who has been previously diagnosed with or identified as suffering from or having a respiratory disorder, one or more complications related to a respiratory disorder, and optionally, but need not have already undergone treatment for such a respiratory disorder.

In some embodiments, the methods of the invention further comprise selecting a subject in need of treatment of an respiratory disorder.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metah. Pharmacokinet, 15(2): 143-53; Balimane and Sinko (1999). Involvement of multiple transporters in the oral absorption of nucleoside analogues, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs-principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS Pharm Sci., 2(1): E6; Sadzuka Y. (2000). Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

To the extent not already indicated, it will be understood by those of ordinary skill in the art that any one of the various embodiments herein described and illustrated may be further modified to incorporate features shown in any of the other embodiments disclosed herein.

The present invention may be defined in any of the following numbered paragraphs:
1. A method of treating a respiratory disorder in a subject, the method comprising inhibiting release of nitric oxide from a basal cell in the subject.
2. The method of paragraph 1, wherein the basal cell is in the upper respiratory tract.
3. The method of any of paragraphs 1-2, wherein the respiratory disorder is an inflammatory disorder of the lung.
4. The method of any of paragraphs 1-3, wherein the respiratory disorder is allergic or non-allergic.
5. The method of any of paragraphs 1-4, wherein the respiratory disorder is selected from the group consisting of atopic asthma, non-atopic asthma, emphysema, bronchitis, chronic obstructive pulmonary disease (COPD), sinusitis, allergic rhinitis.
6. The method of any of paragraphs 1-5, the method comprising administering to the subject a nitric oxide inhibitor.
7. The method of any of paragraphs 1-6, wherein said administering comprises intravenous, transdermal, intrasynovial, intramuscular, oral, or inhalation administration.
8. The method of any of paragraphs 1-7, wherein said administering is inhalation.
9. The method of any of paragraphs 1-8, wherein the nitric oxide inhibitor is a small molecule, peptide, peptide mimetic, protein or a portion thereof, antibody, nucleic acid (e.g. antisense oligonucleotide, siRNA, miRNA, miRNA mimic, antagomir, ribozyme, aptamer, and decoy oligonucleotide), or gene therapy reagent.
10. The method of any of paragraphs 1-9, wherein the nitric oxide inhibitor is selected from the group consisting of 2-phenyl-4,4,5,5tetraethylimidazoline-1-oxyl-3-oxide (PTIO), 2-(4carboxyphenyl)-4,4,5,5-tetraethylimidazoline-1-oxyl-3oxide (Carboxy-PTIO), N-methyl-D-glucamine dithiocarbamate (MOD), N-nitro-L-arginine methyl-ester (L-NAME), N-monomethyl-L-arginine (L-NMMA), 2-ethyl-2-thiopseudourea (ETU), 2-methylisothiourea (SMT), 7-nitroindazole, L-arginine analogues (such as aminoguanidine, aminoguanidine hemisulfate, N-monomethyl-L-arginine, N-nitro-L-arginine, D-arginine and the like) diphenyleneiodonium (DPI), L-cysteine, heparin, SC-51, Vitamin B12 (hydroxocobalamin), cyanocobalamin, hemoglobin, flavohemoglobin, heme, myoglobin, ruthenium (III) polyaminocarboxylate complex, NOX-100, NOX-200, NOX-700, dithiocarbamates which chelate iron to form complex which irreversibly binds free NO including pyrrolidine dithiocarbamate, and esters and prodrugs thereof.
11. The method of any of claims 1-10, wherein nitric oxide inhibitor is linked with a ligand.
12. The method of any of paragraphs 1-11, wherein nitric oxide inhibitor is linked with an antibody against an extracellular epitope of the basal cell.
13. The method of any of claims 1-13, wherein nitric oxide inhibitor is linked with a basal cell specific lectin.

### Examples

### Methods

### Tissue Fixation and Preparation

Adult male Sprague Dawley rats (Charles River Labs, Wilmington, Mass.) were anesthetized with nembutal (60 mg/kg, i.p.). The male reproductive and upper respiratory tracts were fixed by perfusion through the left ventricle with paraformaldehyde-lysine-periodate (PLP) fixative, as described previously (Pastor-Soler et al., 2003). All procedures were approved by the Massachusetts General Hospital Institutional Committee on Research Animal Use.

### In Vivo Microperfusion

Rats were anesthetized and the cauda epididymidis was luminally perfused in vivo, harvested and fixed in PLP, as described previously (Beaulieu et al., 2005; Pastor-Soler et al., 2003; Wong and Yeung, 1978).

### Immunofluorescence

Immunofluorescence labeling was performed on cryostat sections, as described previously (Beaulieu et al., 2005; Pastor-Soler et al., 2003). Primary antibodies, the AGTR2 peptide and secondary antibodies used are listed in supplementary material. Slides were mounted in Vectashield (Vector Labs, Burlingame, Calif.) with or without DAPI. For confocal microscopy, nuclei were stained using TOPRO-3 iodide (Invitrogen, Carlsbad, Calif.). Immunostained sections were examined using a Nikon E800 microscope (Nikon Instruments, Melville, N.Y.). Digital images were acquired with IPLab Spectrum software (Scanalytics, Fairfax, Va.) and imported into Adobe Photoshop. Sections were also examined using a Zeiss Radiance 2000 confocal microscope (Zeiss Laboratories). Z-series (0.1-μm interval) were imported into Volocity software (Improvision Inc., version 4.1) for 3D reconstruction and final animations were exported as Quicktime movies.

### Quantification of V-ATPase Apical Membrane Accumulation in Clear Cells

The level of accumulation of V-ATPase in clear cell microvilli was quantified using IPLab software as described previously (Beaulieu et al., 2005; Pastor-Soler et al., 2003). 10 μm sections of microperfused cauda epididymidis were immunostained under identical conditions, and confocal images were acquired using the same parameters. The segmentation procedure of IPLab was used to measure the area of V-ATPase-positive microvilli, which was normalized against the length of apical pole of each cell (Beaulieu et al., 2005; Pastor-Soler et al., 2003). At least three epididymides from different animals were perfused for each condition, and a minimum of 10 cells/tissue were examined for a total of at least 30 cells/condition.

### Immunogold Electron Microscopy and Quantification of Gold Labeling

Pieces of PLP-fixed epididymis were embedded at −45° C. using HM20 resin (Electron Microscopy Sciences, Hatfield, Pa.) in a Leica EM AFS, and ultrathin sections were cut, as described previously (Da Silva et al., 2007; Pastor-Soler et al., 2003). Sections were immunostained for the V-ATPase A subunit, followed by goat anti-rabbit IgG coupled to 15 nm gold (Ted Pella, Reading, Calif.). Grids were examined in a JEOL 1011 electron microscope. Images were acquired using an AMT digital imaging system.

The number of V-ATPase associated gold particles on the apical membrane and microvilli was counted for each clear cell (Da Silva et al., 2007; Pastor-Soler et al., 2003). To determine the density of V-ATPase molecules along the apical membrane, the number of gold particles was divided by the length of apical membrane, including microvilli, of each cell. This value is referred to as “gold/μm apical membrane”. To determine the relative density of the V-ATPase at the cell surface, the number of gold particles was normalized for the width of the cell, measured at the base of the microvilli (gold/μm cell width).

### Western Blotting

Protein extracts from rat and mouse epididymis were subjected to electrophoresis and western blotting, as described previously (Beaulieu et al., 2005; Pastor-Soler et al., 2003).

### Isolation of Clear Cells, RNA Extraction and RT-PCR

Epididymides from B1-EGFP transgenic mice (Miller et al., 2005) were digested with trypsin and collagenase. Fluorescence-activated cell sorting (FACS) was used to separate clear cells (GFP-positive) from other cell types (GFP-negative). Total RNA was isolated using the PicoPure RNA Isolation kit (Molecular Devices, Sunnyvale, Calif.), and RT-PCR was performed as described previously (Isnard-Bagnis et al., 2003). The primers amplifying a 674-bp fragment of the mouse Agtr2 coding sequence are: attggctttttggacctgtg (MAGTR2-F3) and aaacacactgcggagcttct (MAGTR2-R2). The PCR product was purified with the Qiaquick PCR kit and sequenced by the MGH sequencing core.

### Detection of Proton Secretion

The proximal VD was cut open to expose the apical surface of the epithelium and anchored onto a custom-made chamber. Proton secretion was measured using a self-referencing proton-selective electrode, as described previously (Beaulieu et al., 2005; Breton et al., 1996; Smith et al., 2007).

### Statistical Analysis

The effects of treatments between two groups were determined by paired or unpaired Student's t-test when appropriate. Comparisons between multi-groups were determined by one-way ANOVA with Bonferroni's post-hoc test. All tests were two-tailed and the limit of statistical significance was set at P=0.05.

### Example 1

### Basal Cells Send Long, Slender Cytoplasmic Projections Towards the Lumen

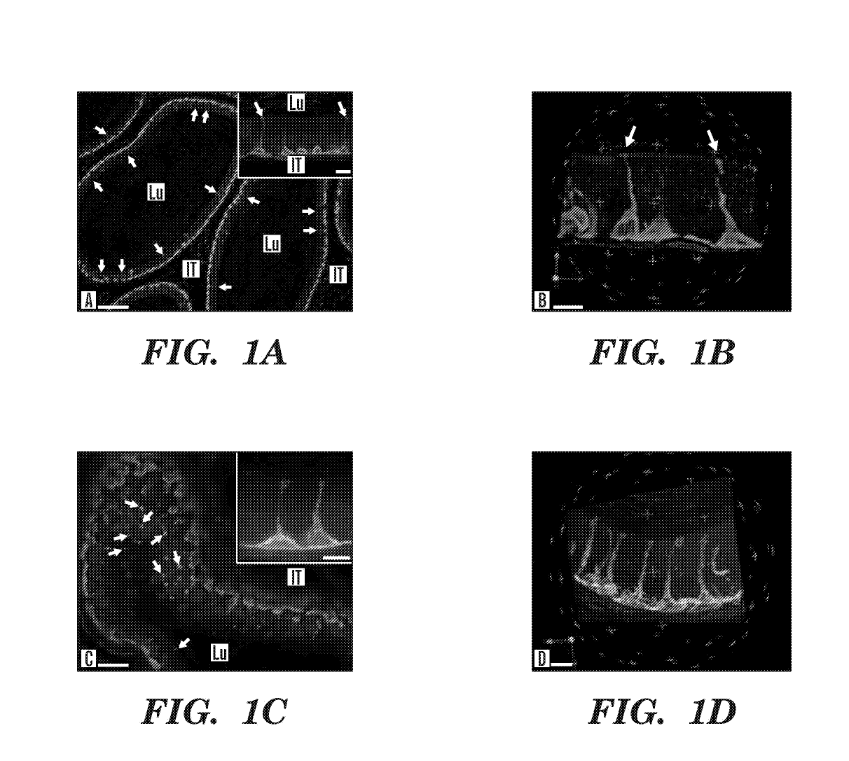

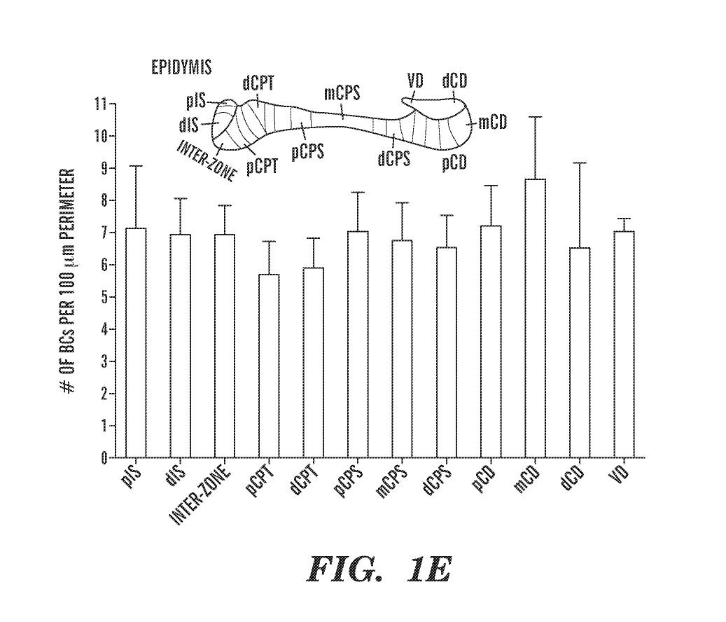

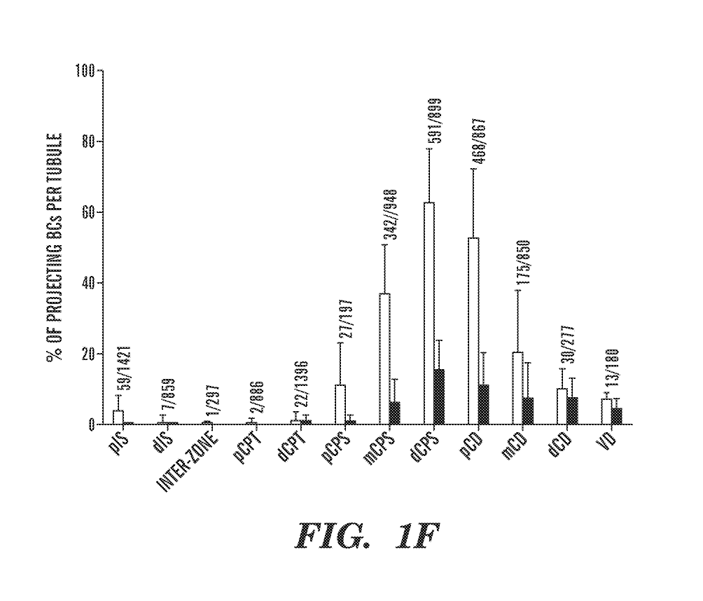

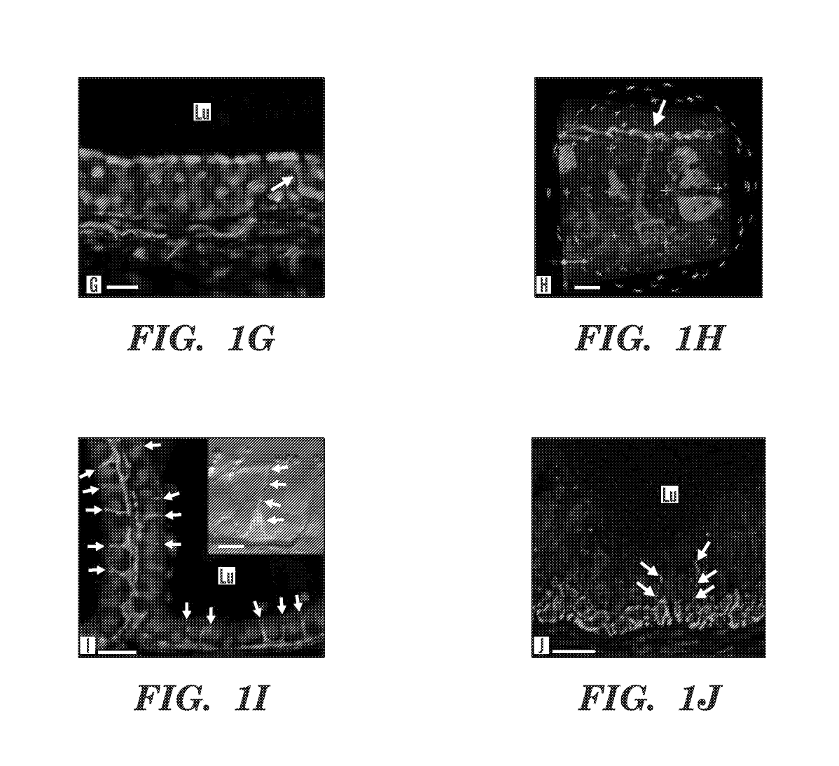

Epididymis sections (16 μm) from rat were labeled for COX1, a marker of basal cells (Leung et al., 2004). While a dense network of basal cells was located at the base of the epithelium confirming previous reports (Clermont and Flannery, 1970; Veri et al., 1993; Yeung et al., 1994), many basal cells exhibited a narrow body extension that infiltrates between other epithelial cells towards the lumen (FIG. 1A: arrows). This was confirmed using 3D-reconstructions from a z-series of confocal images (FIG. 1B). The probability of observing these slender structures in thinner sections, which are more commonly used for staining, and in ultrathin sections used for electron microscopy is low, probably explaining why they have not been described extensively in previous publications. FIG. 1C shows an oblique section stained for claudin-1 (Cldn1, green), another marker of basal cells (Gregory et al., 2001). Numerous projections, positive for Cldn1, were seen between epithelial cells (arrows). Cldn1 is also present at lower levels in the lateral membrane of principal cells (Gregory et al., 2001), but this was not seen under conditions used herein. The inventors did, however, detected basolateral Cldn-1 in principal cells using higher concentrations of antibodies (not shown). The inset in FIG. 1C shows two basal cells double-stained for COX1 (red) and Cldn1 (green) that extend their narrow body towards the lumen. This result was confirmed by 3D reconstruction (FIG. 1D).

A quantitative analysis was performed to determine the number of basal cells reaching the apical pole of the epithelium (defined as the region located above the nuclei of adjacent epithelial cells). Basal cells that projected all the way to the apical border of the epithelium were also counted (FIG. 1F). These numbers were normalized for the total number of basal cell nuclei (FIG. 1E). Individual epididymis regions and the proximal vas deferens (VD) were analyzed separately. While very few basal cells reached the lumen in the proximal regions, the frequency of events progressively increased towards the distal regions, reaching a maximum in the distal corpus and proximal cauda. In the distal cauda and in the vas deferens, fewer basal cells reached the lumen.

Rat trachea sections (16 μm) were labeled for COX1 (red) and tubulin (green), a marker of airway ciliated cells (FIG. 1G). Similarly to the epididymis, some COX1-positive basal cells exhibited a slender projection that extends towards the lumen (arrow). This was confirmed by 3D-reconstruction of sections double-stained for ZO1 (green) and COX1 (red) (FIG. 1H). While the ZO1-labeled tight junctions (TJs) located at the corner between three epithelial cells (tricellular corners) were closed, the TJ located adjacent to the basal cell apical region was partially open. Basal cells with long cytoplasmic projections in contact with the epithelial apical border were also detected in the larynx (data not shown).

FIG. 1I shows that the rat coagulating gland, a tissue morphologically and physiologically analogous to the middle lobe of the human prostate (Price, 1963; Wei et al., 1997), also contained numerous basal cells (stained for COX1 in green; arrows) that send a narrow body projection towards the lumen. The inset is a higher magnification differential interference contrast (DIC) image of a COX1-stained basal cell (green) reaching the luminal border between adjacent epithelial cells (arrows). A dense network of basal cells was also detected in human epididymis stained for Cldn1 (FIG. 1J). Importantly, some basal cell extensions were detected even on “thin” 5 μm sections, indicating that these cells have the capacity to reach the lumen in humans also.

### Example 2

### Basal Cells Cross TJs to Reach the Lumen

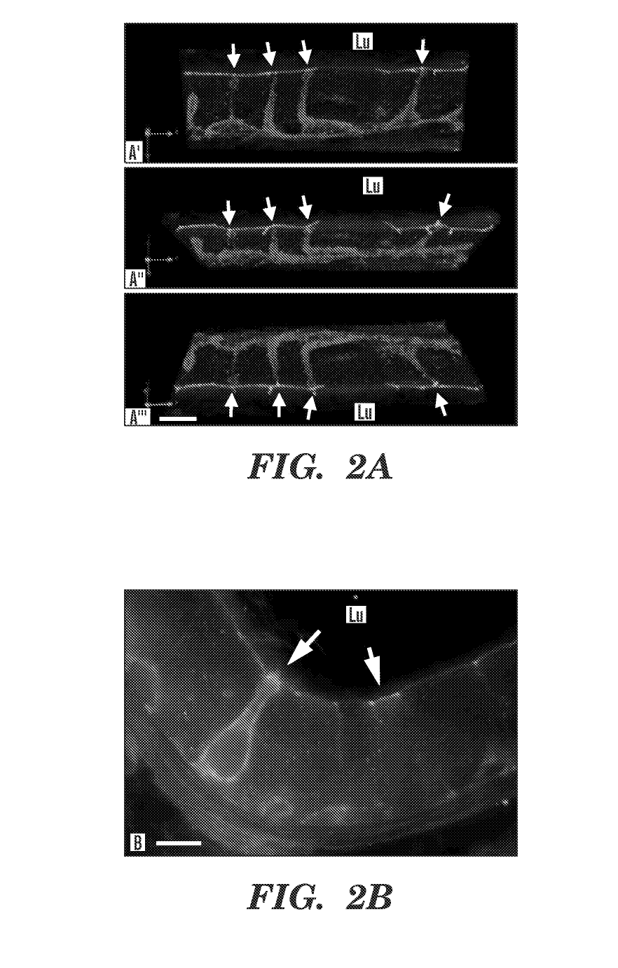

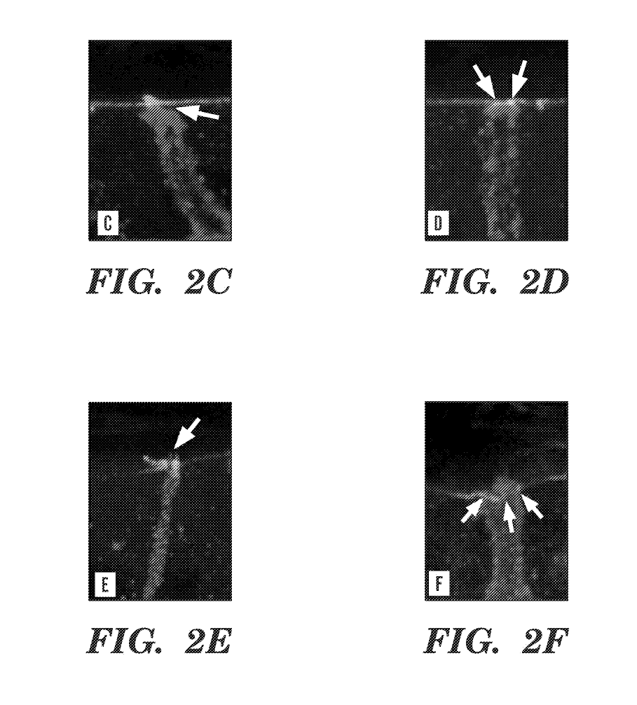

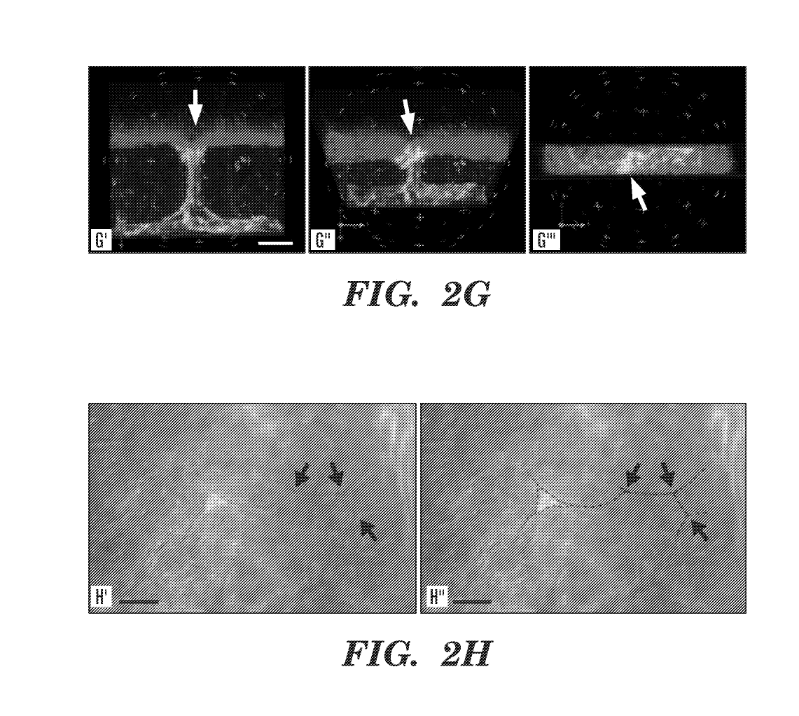

While basal cells have been shown to extend processes between epithelial cells, they have never been disclosed to have direct access to the lumen (Evans et al., 2001; Hermo and Robaire, 2002; Robaire and Viger, 1995; van Leenders and Schalken, 2003; Veri et al., 1993). To determine whether basal cells can cross the TJ barrier, double labeling for Cldn1 and ZO1 was performed on rat epididymis. Basal cells preferentially reach TJs at the tripartite junction between other epithelial cells (FIGS. 2A′, 2A″, and 2A′″, arrows). Various patterns of interaction between basal cells and TJs were seen. FIG. 2B shows a basal cell (arrow) that has crossed the TJ barrier and established contact (labeled with ZO1) with adjacent principal cells. The arrowhead indicates a clear cell with apical V-ATPase labeling (blue). FIGS. 2C-2F are 3D reconstructions of basal cells showing various patterns of interactions with the TJs. FIG. 2C shows one cell underneath the TJ (arrow) but showing no co-localization between Cldn1 and ZO1. The yellow staining in FIG. 2D (arrows) indicates partial co-localization between the Cldn1-positive basal cell and the ZO1-labeled TJ. The tripartite junction adjacent to this basal cell is partially open (data not shown). FIG. 2E shows one cell that crosses the TJ barrier (arrow). FIG. 2F shows one cell that has penetrated the epithelium beyond the TJ barrier and has established contact with adjacent principal cells (arrows, this cell is also shown in FIG. 2B). In the trachea, similar patterns of interaction between basal cells and TJs were also seen, FIG. 1H.

The contact of basal cells with the epididymal lumen was further demonstrated with a marker of principal cell apical stereocilia (F-actin). 3D reconstruction clearly showed that basal cells, stained for Cldn1 (green) but negative for F-actin (arrow), reach the luminal side between principal cells, which are heavily labeled for F-actin (FIGS. 2G′, 2G″, and 2G′″). The luminal contact of this basal cell is apparent only on panels showing rotations around the X axis (FIGS. 2G″ and 2G′″), and is not visible on the XY image shown in FIG. 2G′. This is due to the presence of long stereocilia in adjacent principal cells, which mask the small apical pole of the basal cell. The apical surface of a cut-open epididymal tubule was visualized by DIC coupled to Cldn1 labeling (green) (FIGS. 2H′ and 2H″). While most tripartite cell junctions were closed (arrows), one corner is occupied by a Cldn1-positive basal cell, which has established contact with the lumen.

### Example 3

### Functional Role of Epididymal Basal Cells

The inventor also examined the potential role of the basal cell extensions as sensors/monitors for the presence of biological factors in the lumen. The inventors examined the expression of hormone receptors in these cells. Because luminally located RAS is an important contributor to male fertility, the inventors examined the expression of ANGII receptors in the epididymal epithelium.

### Basal Cells Express AGTR2.

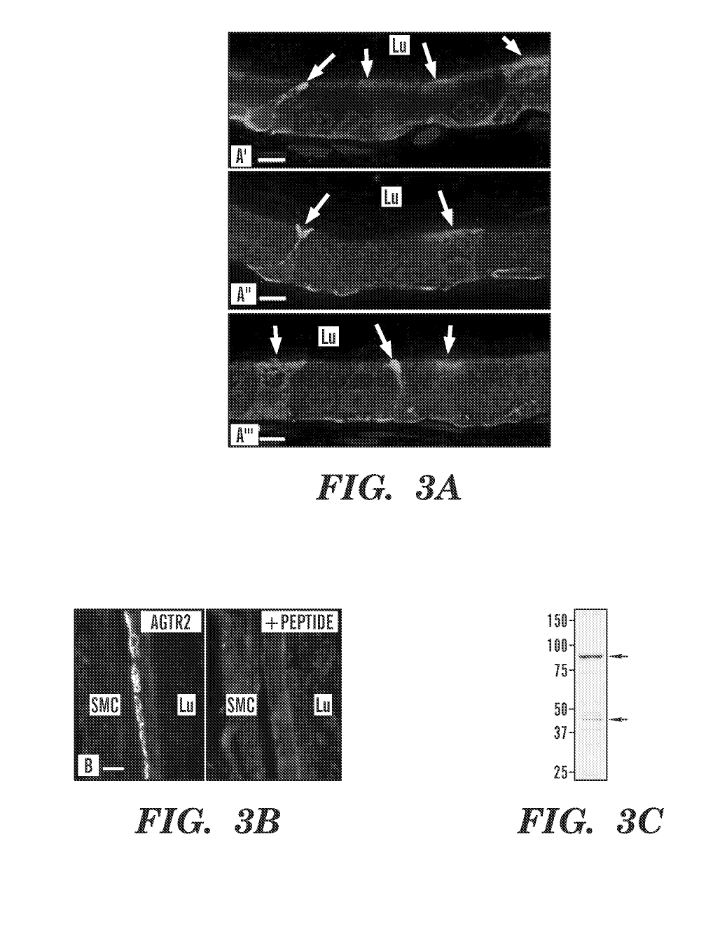

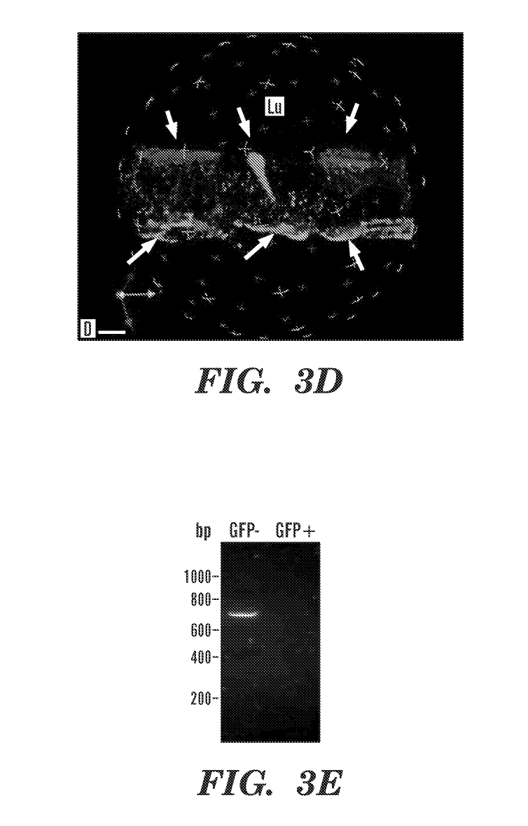

Double-labeling for the proton-pumping V-ATPase (red), located in the apical pole of clear cells (FIG. 3A′-A′″; arrowheads), and AGTR2 (green) showed that AGTR2 was exclusively expressed in basal cells (arrows). This was in agreement with previous studies showing AGTR2 in the basal region of the epithelium, although the cell type expressing AGTR2 was not identified (Leung et al., 1997). AGTR2 staining was abolished when the antibody was pre-absorbed with the immunizing peptide (10-fold excess; FIG. 3B). Western blots of rat epididymis showed two bands, one at about 44 kDa, the expected molecular weight of AGTR2, and a second at about 88 kDa (FIG. 3C; arrows). Both bands were abolished by pre-incubation of antibody with its peptide (not shown). The higher molecular weight band was twice the molecular weight of AGTR2, indicating potential dimerization of AGTR2, as reported for other G-protein coupled receptors (Bulenger et al., 2005; Parnot and Kobilka, 2004; Skrabanek et al., 2007). 3D-reconstruction (FIG. 3D) confirmed that AGTR2 was expressed in basal cells (arrows). Two clear cells stained at their apical pole for the V-ATPase (arrowheads), but negative for AGTR2, were located close to basal cells. The absence of AGTR2 from clear cells was further confirmed by RT-PCR using B1-EGFP transgenic mice in which enhanced GFP is expressed only in clear cells (Miller et al., 2005). Clear cells isolated by fluorescence activated cell sorting (FACS) were compared to GFP-negative cells, i.e. all other cell types in the epididymis. Whereas a positive signal was obtained using primer sets spanning the coding region of agtr2 in non-clear cells (FIG. 3E: GFP−) no signal was detected in clear cells (GFP+). The identity of the PCR product was confirmed by direct sequencing (not shown).

### Basal Cells Sense Luminal ANGII and Regulate Clear Cells Via AGTR2

In the kidney, ANGII stimulates proton secretion by intercalated cells (Pech et al., 2008; Rothenberger et al., 2007), which resemble epididymal clear cells (Breton and Brown, 2007). The expression of AGTR2 exclusively in basal cells can allow these cells to regulate proton secretion by clear cells, following sampling of luminal ANGII. The inventors had previously shown that V-ATPase apical membrane accumulation and extension of microvilli in clear cells correlate with proton secretion (Beaulieu et al., 2005; Pastor-Soler et al., 2003). Here, the inventors examined the effect of ANGII on the extension of V-ATPase-labeled microvilli.

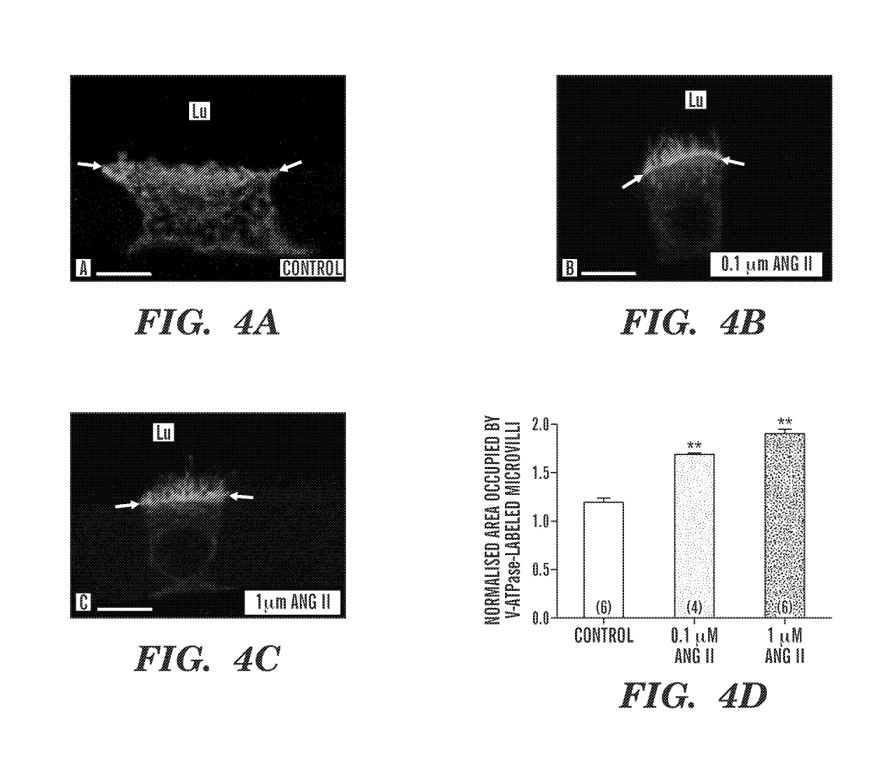

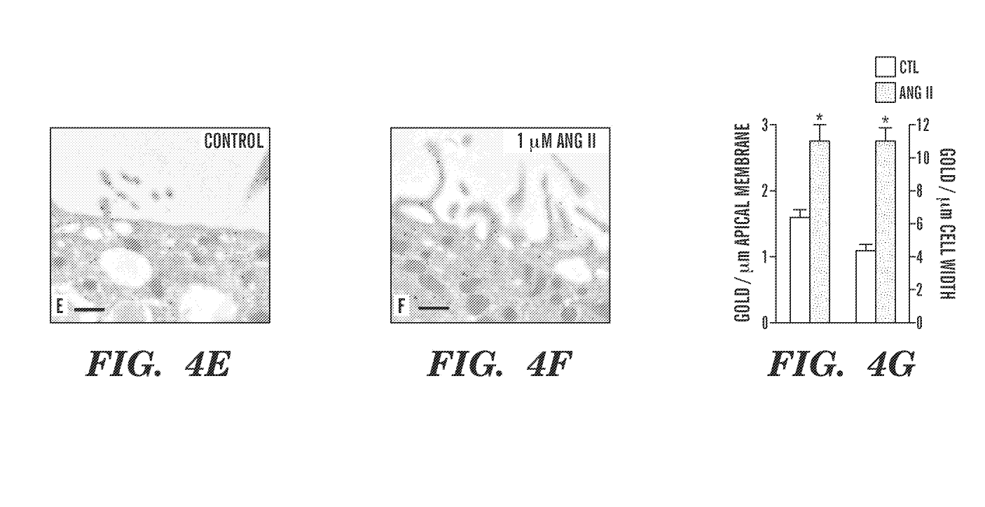

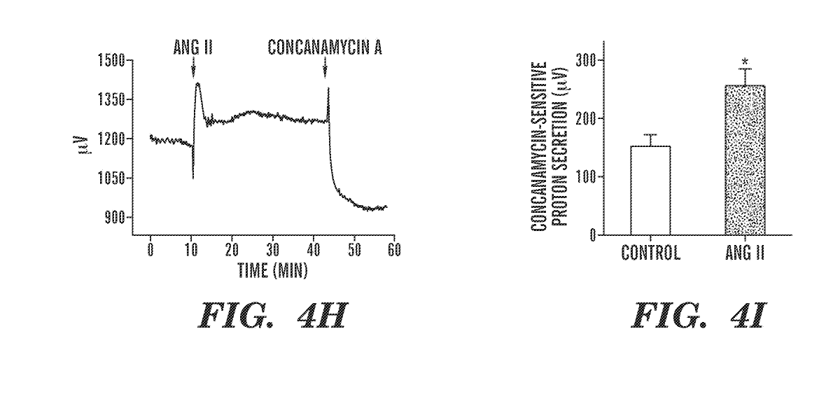

Rat cauda epididymides were perfused luminally in vivo with phosphate-buffered saline (pH 6.6). Under these control conditions, clear cell V-ATPases were distributed between short microvilli and sub-apical vesicles (FIG. 4A). Addition of ANGII (0.1 and 1 μM) to the luminal perfusate significantly increased the extension of V-ATPase-labeled microvilli to 141±4% and 153±7% versus control, respectively (FIGS. 4B-D). Immunogold electron microscopy confirmed this accumulation of V-ATPase in apical microvilli (FIGS. 4E-G). ANGII induced a significant increase in the density of V-ATPase molecules in microvilli (FIG. 4G, Gold/μm apical membrane). Because numerous and longer microvilli were observed in clear cells exposed to ANGII, the total number of V-ATPase molecules located at the cell surface was further amplified compared to control (FIG. 4G, Gold/μm cell width).

The effect of ANGII on proton secretion was examined in cut-open proximal VD (FIG. 4H), a tissue that also contained clear and basal cells, using an extracellular proton-selective microelectrode (Beaulieu et al., 2005; Breton et al., 1996). After a control period during which stable proton secretion was recorded, ANGII was added to the bath. After a rapid and transient rise due to disturbance of the proton gradient, proton secretion showed a sustained increase. Addition of the V-ATPase inhibitor concanamycin A markedly inhibited proton secretion. For each VD, both the control value (prior to addition of ANGII) and the ANGII value (30 min after its addition) were corrected for the value measured after addition of concanamycin A. On average, ANGII caused a significant increase of concanamycin-sensitive proton secretion of 68% compared to control (FIG. 4I). Pre-incubation of the tissue with concanamycin A for 10 min prevented the response to ANGII (data not shown).

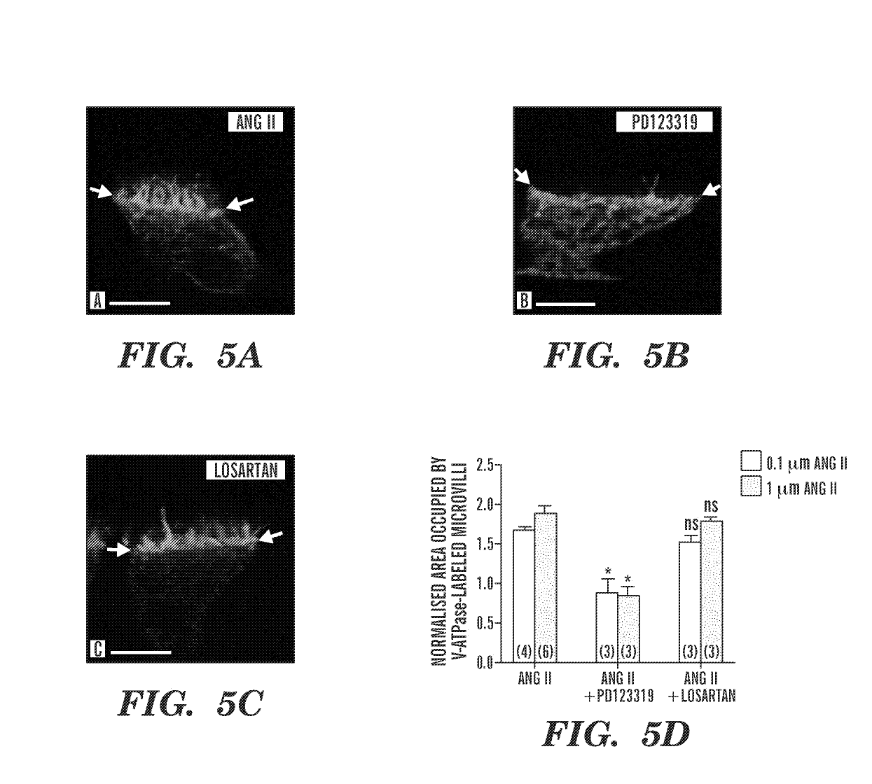

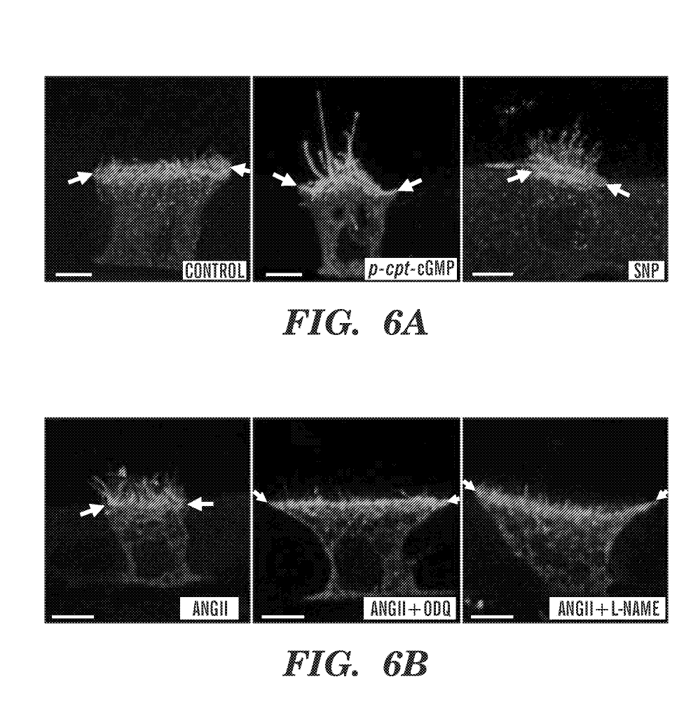

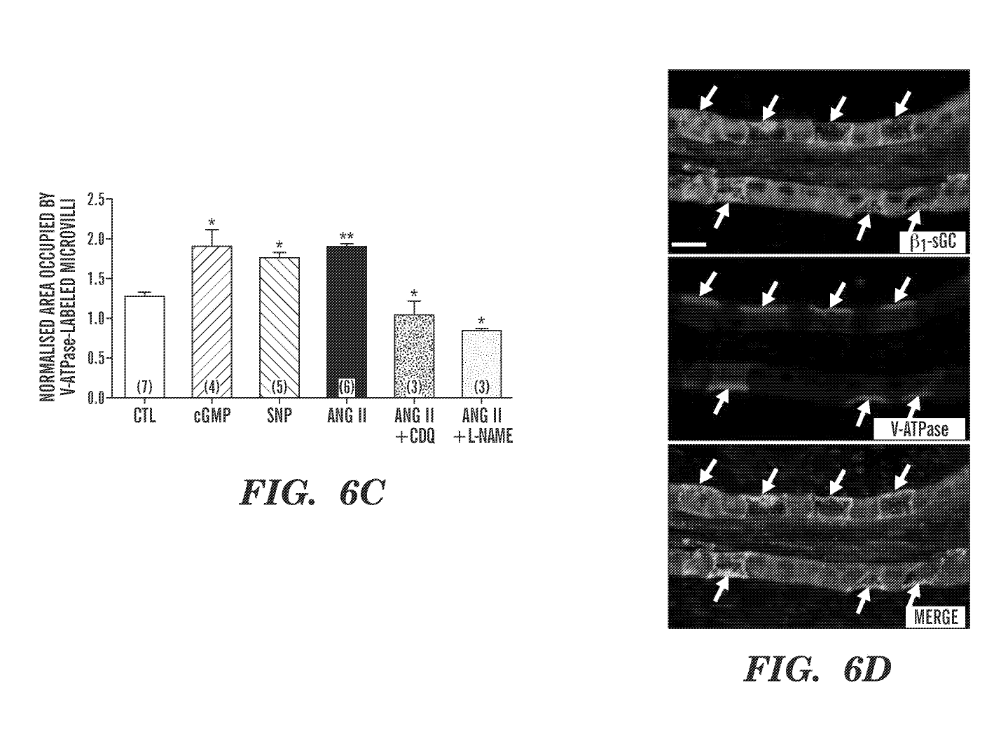

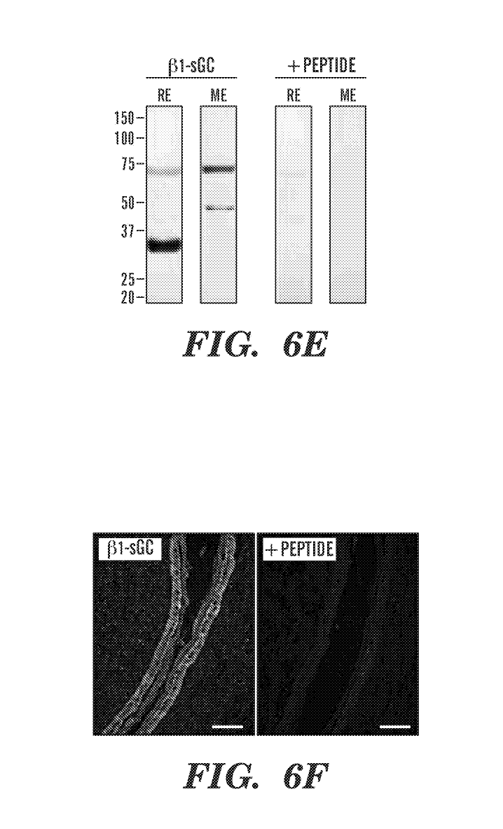

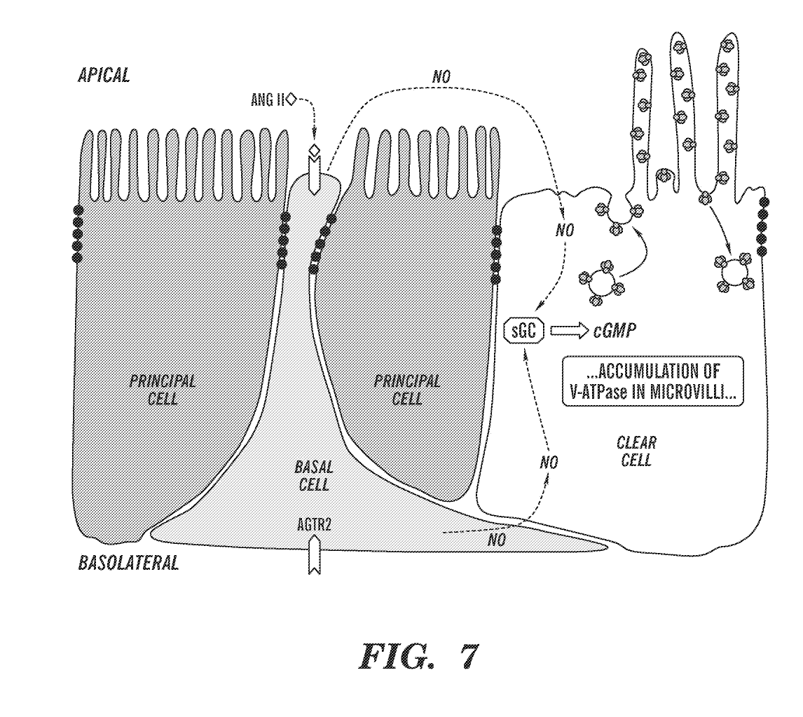

Losartan, an AGTR1 antagonist, had no inhibitory effect on ANGII-induced V-ATPase apical accumulation (FIGS. 5C and 5D). However the AGTR2 antagonist, PD123319, prevented the stimulatory effect of ANGII on clear cells (FIGS. 5B and 5D). These results were consistent with participation of AGTR2 in the regulation of clear cell-dependent luminal acidification. Nitric oxide (NO) is the downstream effector of AGTR2 activation (Carey, 2005; Toda et al., 2007). p-cpt-cGMP, a cell-permeable analogue of cGMP, or sodium nitroprusside (SNP), a NO-donor, induced a significant elongation of V-ATPase-rich microvilli, compared to control (FIGS. 6A and 6C). Pretreatment with the soluble guanylate cyclase (sGC) inhibitor ODQ, or the NO synthase (NOS) inhibitor L-NAME, completely abolished ANGII-induced V-ATPase apical accumulation (FIGS. 6B and 6C). Immunofluorescence labeling showed a strong staining for the β₁ subunit of sGC (β1-sGC) in the basolateral membrane and apical region of clear cells (FIG. 6D: green), identified by apical staining for the V-ATPase (red). Specificity of the antibody was confirmed by Western blot and immunofluorescence using antibody that had been pre-absorbed with β₁-sGC peptide (FIGS. 6E and 6F).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

### REFERENCES

1. Beaulieu, V., Da Silva, N., Pastor-Soler, N., Brown, C. R., Smith, P. J., Brown, D., and Breton, S. (2005). Modulation of the actin cytoskeleton via gelsolin regulates vacuolar H+-ATPase recycling. J Biol Chem 280, 8452-8463.
2. Blomqvist, S. R., Vidarsson, H., Soder, O., and Enerback, S. (2006). Epididymal expression of the forkhead transcription factor Foxil is required for male fertility. Embo J 25, 4131-4141.
3. Breton, S., and Brown, D. (2007). New insights into the regulation of V-ATPase-dependent proton secretion. Am J Physiol Renal Physiol 292, F1-10.
4. Breton, S., Smith, P. J., Lui, B., and Brown, D. (1996). Acidification of the male reproductive tract by a proton pumping (H+)-ATPase. Nat Med 2, 470-472.
5. Brown, D., Lui, B., Gluck, S., and Sabolic, I. (1992). A plasma membrane proton ATPase in specialized cells of rat epididymis. Am J Physiol 263, C913-916.
6. Bulenger, S., Marullo, S., and Bouvier, M. (2005). Emerging role of homo- and heterodimerization in G-protein-coupled receptor biosynthesis and maturation. Trends Pharmacol Sci 26, 131-137.
7. Burns, A. R., Bowden, R. A., MacDonell, S. D., Walker, D. C., Odebunmi, T. O., Donnachie, E. M., Simon, S. I., Entman, M. L., and Smith, C. W. (2000). Analysis of tight junctions during neutrophil transendothelial migration. J Cell Sci 113 (Pt 1), 45-57.
8. Carey, R. M. (2005). Update on the role of the AT2 receptor. Curr Opin Nephrol Hypertens 14, 67-71.
9. Cheung, K. H., Leung, G. P., Leung, M. C., Shum, W. W., Zhou, W. L., and Wong, P. Y. (2005). Cell-cell interaction underlies formation of fluid in the male reproductive tract of the rat. J Gen Physiol 125, 443-454.
10. Clermont, Y., and Flannery, J. (1970). Mitotic activity in the epithelium of the epididymis in young and old adult rats. Biol Reprod 3, 283-292.
11. Da Silva, N., Shum, W. W., El-Annan, J., Paunescu, T. G., McKee, M., Smith, P. J., Brown, D., and Breton, S. (2007). Relocalization of the V-ATPase B2 subunit to the apical membrane of epididymal clear cells of mice deficient in the B1 subunit. Am J Physiol Cell Physiol 293, C199-210.
12. Esther, C. R., Jr., Howard, T. E., Marino, E. M., Goddard, J. M., Capecchi, M. R., and Bernstein, K. E. (1996). Mice lacking angiotensin-converting enzyme have low blood pressure, renal pathology, and reduced male fertility. Lab Invest 74, 953-965.
13. Evans, M. J., Van Winkle, L. S., Fanucchi, M. V., and Plopper, C. G. (2001). Cellular and molecular characteristics of basal cells in airway epithelium. Exp Lung Res 27, 401-415.
14. Ford, J. R., and Terzaghi-Howe, M. (1992). Basal cells are the progenitors of primary tracheal epithelial cell cultures. Exp Cell Res 198, 69-77.
15. Gatti, J. L., Druart, X., Guerin, Y., Dacheux, F., and Dacheux, J. L. (1999). A 105- to 94-kilodalton protein in the epididymal fluids of domestic mammals is angiotensin I-converting enzyme (ACE); evidence that sperm are the source of this ACE. Biol Reprod 60, 937-945.
16. Gregory, M., and Cyr, D. G. (2006). Identification of multiple claudins in the rat epididymis. Mol Reprod Dev 73, 580-588.
17. Gregory, M., Dufresne, J., Hermo, L., and Cyr, D. (2001). Claudin-1 is not restricted to tight junctions in the rat epididymis. Endocrinology 142, 854-863.
18. Hagaman, J. R., Moyer, J. S., Bachman, E. S., Sibony, M., Magyar, P. L., Welch, J. E., Smithies, O., Krege, J. H., and O'Brien, D. A. (1998). Angiotensin-converting enzyme and male fertility. Proc Natl Acad Sci USA 95, 2552-2557.
19. Hajj, R., Baranek, T., Le Naour, R., Lesimple, P., Puchelle, E., and Coraux, C. (2007). Basal cells of the human adult airway surface epithelium retain transit-amplifying cell properties. Stem Cells 25, 139-148.
20. Hermo, L., and Robaire, B. (2002). Epididymal cell types and their functions, In The Epididymis: From Molecules to Clinical Practice, B. Robaire, and B. T. Hinton, eds. (New York: Kluwer Acedemic/Plenum Publishers), pp. 81-102.
21. Hinton, B. T., and Palladino, M. A. (1995). Epididymal epithelium: its contribution to the formation of a luminal fluid microenvironment. Microsc Res Tech 30, 67-81.
22. Ihrler, S., Zietz, C., Sendelhofert, A., Lang, S., Blasenbreu-Vogt, S., and Lohrs, U. (2002). A morphogenetic concept of salivary duct regeneration and metaplasia. Virchows Arch 440, 519-526.
23. Isnard-Bagnis, C., Da Silva, N., Beaulieu, V., Yu, A. S., Brown, D., and Breton, S. (2003). Detection of C1C-3 and C1C-5 in epididymal epithelium: immunofluorescence and RT-PCR after LCM. Am J Physiol Cell Physiol 284, C220-232.
24. Jones, R. C., and Murdoch, R. N. (1996). Regulation of the motility and metabolism of spermatozoa for storage in the epididymis of eutherian and marsupial mammals. Reprod Fertil Dev 8, 553-568.
25. Kondoh, G., Tojo, H., Nakatani, Y., Komazawa, N., Murata, C., Yamagata, K., Maeda, Y., Kinoshita, T., Okabe, M., Taguchi, R., and Takeda, J. (2005). Angiotensin-converting enzyme is a GPI-anchored protein releasing factor crucial for fertilization. Nat Med 11, 160-166.
26. Krege, J. H., John, S. W., Langenbach, L. L., Hodgin, J. B., Hagaman, J. R., Bachman, E. S., Jennette, J. C., O'Brien, D. A., and Smithies, O. (1995). Male-female differences in fertility and blood pressure in ACE-deficient mice. Nature 375, 146-148.
27. Lavker, R. M., Tseng, S. C., and Sun, T. T. (2004). Corneal epithelial stem cells at the limbus: looking at some old problems from a new angle. Exp Eye Res 78, 433-446.
28. Leung, C. T., Coulombe, P. A., and Reed, R. R. (2007). Contribution of olfactory neural stem cells to tissue maintenance and regeneration. Nat Neurosci 10, 720-726.
29. Leung, G. P., Cheung, K. H., Leung, C. T., Tsang, M. W., and Wong, P. Y. (2004). Regulation of epididymal principal cell functions by basal cells: role of transient receptor potential (Trp) proteins and cyclooxygenase-1 (COX-1). Mol Cell Endocrinol 216, 5-13.
30. Leung, P. S., Chan, H. C., Fu, L. X., Zhou, W. L., and Wong, P. Y. (1997). Angiotensin II receptors, AT1 and AT2 in the rat epididymis. Immunocytochemical and electrophysiological studies. Biochim Biophys Acta 1357, 65-72.
31. Leung, P. S., and Sernia, C. (2003). The renin-angiotensin system and male reproduction: new functions for old hormones. J Mol Endocrinol 30, 263-270.
32. Mewe, M., Bauer, C. K., Muller, D., and Middendorff, R. (2006). Regulation of spontaneous contractile activity in the bovine epididymal duct by cyclic guanosine 5′-monophosphate-dependent pathways. Endocrinology 147, 2051-2062.
33. Miller, R. L., Zhang, P., Smith, M., Beaulieu, V., Paunescu, T. G., Brown, D., Breton, S., and Nelson, R. D. (2005). V-ATPase B1-subunit promoter drives expression of EGFP in intercalated cells of kidney, clear cells of epididymis and airway cells of lung in transgenic mice. Am J Physiol Cell Physiol 288, C1134-1144.
34. Niess, J. H., Brand, S., Gu, X., Landsman, L., Jung, S., McCormick, B. A., Vyas, J. M., Boes, M., Ploegh, H. L., Fox, J. G., et al. (2005). CX3CR1-mediated dendritic cell access to the intestinal lumen and bacterial clearance. Science 307, 254-258.
35. Parnot, C., and Kobilka, B. (2004). Toward understanding GPCR dimers. Nat Struct Mol Biol 11, 691-692.
36. Pastor-Soler, N., Beaulieu, V., Litvin, T. N., Da Silva, N., Chen, Y., Brown, D., Buck, J., Levin, L. R., and Breton, S. (2003). Bicarbonate-regulated adenylyl cyclase (sAC) is a sensor that regulates pH-dependent V-ATPase recycling. J Biol Chem 278, 49523-49529.
37. Pastor-Soler, N., Hallows, K. R., Smolak, C., Gong, F., Brown, D., and Breton, S. (2008). Alkaline pH- and cAMP-induced V-ATPase membrane accumulation is mediated by protein kinase A in epididymal clear cells. Am J Physiol Cell Physiol 294, C488-C494.
38. Pastor-Soler, N., Pietrement, C., and Breton, S. (2005). Role of acid/base transporters in the male reproductive tract and potential consequences of their malfunction. Physiology (Bethesda) 20, 417-428.
39. Pech, V., Zheng, W., Pham, T. D., Verlander, J. W., and Wall, S. M. (2008). Angiotensin II activates H+-ATPase in type A intercalated cells. J Am Soc Nephrol 19, 84-91.
40. Price, D. (1963). Comparative Aspects Of Development And Structure In The Prostate. Natl Cancer Inst Monogr 12, 1-27.
41. Rescigno, M., Urbano, M., Valzasina, B., Francolini, M., Rotta, G., Bonasio, R., Granucci, F., Kraehenbuhl, J. P., and Ricciardi-Castagnoli, P. (2001). Dendritic cells express tight junction proteins and penetrate gut epithelial monolayers to sample bacteria. Nat Immunol 2, 361-367.
42. Rizzo, S., Attard, G., and Hudson, D. L. (2005). Prostate epithelial stem cells. Cell Prolif 38, 363-374.
43. Robaire, B., and Viger, R. S. (1995). Regulation of epididymal epithelial cell functions. Biol Reprod 52, 226-236.
44. Rothenberger, F., Velic, A., Stehberger, P. A., Kovacikova, J., and Wagner, C. A. (2007). Angiotensin II stimulates vacuolar H+-ATPase activity in renal acid-secretory intercalated cells from the outer medullary collecting duct. J Am Soc Nephrol 18, 2085-2093.
45. Saez, F., Legare, C., Laflamme, J., and Sullivan, R. (2004). Vasectomy-dependent dysregulation of a local renin-angiotensin system in the epididymis of the cynomolgus monkey (Macaca fascicularis). J Androl 25, 784-796.
46. Schneeberger, E. E., and Lynch, R. D. (2004). The tight junction: a multifunctional complex. Am J Physiol Cell Physiol 286, C1213-1228.
47. Skrabanek, L., Murcia, M., Bouvier, M., Devi, L., George, S. R., Lohse, M. J., Milligan, G., Neubig, R., Palczewski, K., Parmentier, M., et al. (2007). Requirements and ontology for a G protein-coupled receptor oligomerization knowledge base. BMC Bioinformatics 8, 177.
48. Smith, P. J. S., Sanger, R. S., and Messerli, M. A. (2007). Principles, development and applications of self-referencing electrochemical microelectrodes to the determination of fluxes at cell membranes, In Methods and new frontiers in neuroscience, A. C. Michael, ed. (CRC Press), pp. 373-406.
49. Stein, B., Khew-Goodall, Y., Gamble, J., and Vadas, M. A. (1997). Transmigration of leukocytes, In Endothelium in Clinical Practice: Source and Target of Novel Therapies, G. M. Rubanyi, and V. J. Dzau, eds. (New York: Marcel Dekker, Inc.), pp. 147-202.
50. Takano, K., Kojima, T., Go, M., Murata, M., Ichimiya, S., Himi, T., and Sawada, N. (2005). HLA-DR- and CD11c-positive dendritic cells penetrate beyond well-developed epithelial tight junctions in human nasal mucosa of allergic rhinitis. J Histochem Cytochem 53, 611-619.
51. Toda, N., Ayajiki, K., and Okamura, T. (2007). Interaction of endothelial nitric oxide and angiotensin in the circulation. Pharmacol Rev 59, 54-87.
52. van Leenders, G. J., and Schalken, J. A. (2003). Epithelial cell differentiation in the human prostate epithelium: implications for the pathogenesis and therapy of prostate cancer. Crit Rev Oncol Hematol 46 Suppl, S3-10.
53. Veri, J. P., Hermo, L., and Robaire, B. (1993). Immunocytochemical localization of the Yf subunit of glutathione S-transferase P shows regional variation in the staining of epithelial cells of the testis, efferent ducts, and epididymis of the male rat. J Androl 14, 23-44.
54. Wei, C., Willis, R. A., Tilton, B. R., Looney, R. J., Lord, E. M., Barth, R. K., and Frelinger, J. G. (1997). Tissue-specific expression of the human prostate-specific antigen gene in transgenic mice: implications for tolerance and immunotherapy. Proc Natl Acad Sci USA 94, 6369-6374.
55. Wong, P. Y., and Yeung, C. H. (1978). Absorptive and secretory functions of the perfused rat cauda epididymidis. J Physiol 275, 13-26.
56. Yeung, C. H., Nashan, D., Sorg, C., Oberpenning, F., Schulze, H., Nieschlag, E., and Cooper, T. G. (1994). Basal cells of the human epididymis—antigenic and ultrastructural similarities to tissue-fixed macrophages. Biol Reprod 50, 917-926.
57. Zini, A., O'Bryan, M. K., Magid, M. S., and Schlegel, P. N. (1996). Immunohistochemical localization of endothelial nitric oxide synthase in human testis, epididymis, and vas deferens suggests a possible role for nitric oxide in spermatogenesis, sperm maturation, and programmed cell death. Biol Reprod 55, 935-941.

What is claimed:

1. A method of treating a respiratory disorder in a subject, the method comprising inhibiting release of nitric oxide from a basal cell in the subject.

2. The method of claim 1, wherein the basal cell is in the upper respiratory tract.

3. The method of claim 1, wherein the respiratory disorder is an inflammatory disorder of the lung.

4. The method of claim 3, wherein the respiratory disorder is allergic or non-allergic.

5. The method of claim 4, wherein the respiratory disorder is selected from the group consisting of atopic asthma, non-atopic asthma, emphysema, bronchitis, chronic obstructive pulmonary disease (COPD), sinusitis, allergic rhinitis.

6. The method of claim 2, the method comprising administering to the subject a nitric oxide inhibitor.

7. The method of claim 6, wherein said administering comprises intravenous, transdermal, intrasynovial, intramuscular, oral, or inhalation administration.

8. The method of claim 7, wherein said administering is inhalation.

9. The method of claim 6, wherein the nitric oxide inhibitor is a small molecule, peptide, peptide mimetic, protein or a portion thereof, antibody, nucleic acid (e.g. antisense oligonucleotide, siRNA, miRNA, miRNA mimic, antagomir, ribozyme, aptamer, and decoy oligonucleotide), or gene therapy reagent.

10. The method of claim 9, wherein the nitric oxide inhibitor is selected from the group consisting of 2-phenyl-4,4,5,5tetraethylimidazoline-1-oxyl-3-oxide (PTIO), 2-(4carboxyphenyl)-4,4,5,5-tetraethylimidazoline-1-oxyl-3oxide (Carboxy-PTIO), N-methyl-D-glucamine dithiocarbamate (MOD), N-nitro-L-arginine methyl-ester (L-NAME), N-monomethyl-L-arginine (L-NMMA), 2-ethyl-2-thiopseudourea (ETU,), 2-methylisothiourea (SMT), 7-nitroindazole, L-arginine analogues (such as aminoguanidine, aminoguanidine hemisulfate, N-monomethyl-L-arginine, N-nitro-L-arginine, D-arginine and the like) diphenyleneiodonium (DPI), L-cysteine, heparin, SC-51, Vitamin B12 (hydroxocobalamin), cyanocobalamin, hemoglobin, flavohemoglobin, heme, myoglobin, ruthenium (III) polyaminocarboxylate complex, NOX-100, NOX-200, NOX-700, dithiocarbamates which chelate iron to form complex which irreversibly binds free NO including pyrrolidine dithiocarbamate, and esters and prodrugs thereof.

11. The method of claim 9, wherein nitric oxide inhibitor is linked with an antibody against an extracellular epitope of the basal cell.
